SEQUENCE LISTING (4 sequences)

SEQ ID NO: 1 - DNA, 20 bp, synthetic construct
  source 1..20: mol_type = other DNA
accacagtccatgccatcac

SEQ ID NO: 2 - DNA, 20 bp, synthetic construct
  source 1..20: mol_type = other DNA
tccaccaccctgttgctgta

SEQ ID NO: 3 - DNA, 21 bp, synthetic construct
  source 1..21: mol_type = other DNA
acggtcaggtcatcactatcg

SEQ ID NO: 4 - DNA, 22 bp, synthetic construct
  source 1..22: mol_type = other DNA
tgccacaggattccatacccag

(19) United States
(12) Patent Application Publication
(10) Pub. No.: US 2025/0073342 A1
(43) Pub. Date: Mar. 6, 2025

(54) HYPERBRANCHED POLY(BETA-AMINO ESTER) FOR SIRNA DELIVERY AND GENE SILENCING

(71) Applicant: Clearlab SG Pte Ltd., Singapore (SG)

(72) Inventors: Ying Jie Ooi, Singapore (SG); Jong Gu Park, Singapore (SG); Mary B. Chan-Park, Singapore (SG)

(21) Appl. No.: 18/815,270

(22) Filed: Aug. 26, 2024

Related U.S. Application Data
(60) Provisional application No. 63/578,567, filed on Aug. 24, 2023.

(51) Int. Cl. A61K 47/59; A61K 47/69; C08G 63/685; C08G 73/02; C12N 15/113

(52) U.S. Cl. CPC A61K 47/593; A61K 47/6931; C08G 73/0233; C12N 15/113; C12N 2310/14

(57) ABSTRACT

Provided herein are hyperbranched poly(β-amino esters) polymers that serve as effective transfection carriers of small interfering RNA (siRNA) for RNA interference (RNAi) mediated gene silencing therapy. These disclosed polymers exhibit outstanding gene silencing efficiency in both easy-to-transfect and hard-to-transfect cells. Furthermore, they are biodegradable and non-toxic.

### CROSS-REFERENCE TO RELATED APPLICATION

This application claims the benefit of U.S. Provisional Application 63/578,567, filed Aug. 24, 2023, the contents of which are hereby incorporated in its entirety.

### REFERENCE TO SEQUENCE LISTING

The Sequence Listing submitted Aug. 26, 2024, as a text file named “11073-003W01_ST26.txt” created on Aug. 26, 2024, and having a size of 4,560 bytes in size is hereby incorporated by reference pursuant to 37 C.F.R. § 1.52(e)(5).

### FIELD OF THE DISCLOSURE

The disclosure relates to hyperbranched polymers, which can be used as transfection carrier of small interfering RNA (siRNA) for RNA interference (RNAi) mediated gene silencing therapy. The disclosure not only provides efficient siRNA delivery and excellent gene silencing, but also low cytotoxicity and biodegradable.

### BACKGROUND

The listing or discussion of a document in this specification should not necessarily be taken as an acknowledgement that the document is part of the state of the art or is common general knowledge.

RNA interference (RNAi) mediated gene silencing is a biological cellular pathway that targets the messenger RNAs to knockdown the RNA expression in a sequence-specific manner (Agrawal et al., Microbiol. Mol. Biol. Rev. 2003, 67, 657-85; Svoboda, Front Plant Sci. 2020, 11). This pathway can be externally induced by intracellular delivery of 21- to 23-nucleotide double stranded RNA, also known as small interfering RNA (siRNA), which can specifically recognize its homologous mRNAs sequence and direct the degradation of the mRNAs (Chi et al., Proc. Natl. Acad. Sci. 2003, 100, 6343-6346; Fire et al., Nature 1998, 391, 806-811). Targeted gene silencing via siRNA delivery holds great potential for the treatment of various diseases such as viral infections, cancer, and neurodegenerative disorders (Karagiannis & El-Osta, Cancer Gene Ther. 2005, 12, 787-795). However, the therapeutic approach based on siRNA delivery cannot be achieved without a safe and efficient delivery vector, which remains a central challenge to its therapeutic application (Whitehead, Langer, & Anderson, Nat. Rev. Drug Discov. 2009, 8, 129-138).

Non-viral gene vectors such as cationic polymers have been widely used as delivery carriers for siRNA delivery because of their safety profile, low-cost, large-scale manufacturing potential, stability, and capacity for larger nucleic acid payload (Ediriwickrema & Saltzman, ACS Biomater. Sci. Eng. 2015, 1, 64-78; Yin et al., Nat. Rev. Genet. 2014, 15, 541-555). Cationic polymers, such as polyethylenimine (PEI) (Helmschrodt et al., Mol. Ther. Nucleic Acids 2017, 9, 57-68; F. Liu et al., Int. J. Mol. Sci. 2022, 23, 5014; Lu, Morris, & Labhasetwar, J. Pharmacol. Exp. Ther. 2019, 370, 902-910; Malek, Czubayko, & Aigner, J. Drug Target. 2008, 16, 124-139), poly-L-lysine (PLL) (Alazzo et al., J. Mater. Chem. 2022, 10, 236-246; Inoue et al., J. Control. Release. 2008, 126, 59-66), and poly(2-dimethylaminoethyl methacrylate) (pDMAEMA) (Kong et al., J. Control. Release. 2009, 138 (2), 141-7; Li, Sharili, Connelly, & Gautrot, Biomacromolecules 2018, 19, 606-615) can spontaneously condense negatively charged RNA into complexes with sizes of around 50-200 nm and positive surface charge which in turn enhance the cellular uptake via charged-mediated endocytosis. Although showing good transfection efficiency, these early polymers are non-biodegradable which dramatically limits their application in the therapeutic field. Therefore, it is necessary to develop a biodegradable gene delivery system to ensure a good safety window.

Poly(β-amino esters) (pBAEs) is a class of biodegradable cationic polymers which have great potential and high transfection efficiency for DNA and siRNA delivery (Dosta et al., Cardiovasc. Eng. Technol. 2021, 12, 114-125; Dosta, Ramos, & Borrós, Mol. Syst. Des. Eng. 2018, 3, 677-689). A variety of linear pBAEs with different combinations of amine and diacrylate monomers has been developed for gene delivery applications with various cell types (Kamat et al., Mol. Cancer Ther. 2013, 12, 405-15; Mangraviti et al., ACS Nano 2015, 9, 1236-1249; Tzeng et al., Biomaterials 2011, 32, 5402-10; Tzeng, Higgins, Pomper, & Green, J. Biomed. Mater. Res.-A 2013, 101, 1837-45). However, linear structures contain only two terminal units and limited cationic charge center, hence limiting the chemical space for functionalization and development of gene delivery vector with different architectures. The transition from linear to highly branched pBAEs can significantly improve the transfection efficiency on a variety of cell lines, compared to their corresponding linear structure (Cutlar et al., Biomacromolecules 2015, 16, 2609-2617; Gao et al., J. Control. Release. 2020, 321, 654-668; S. Liu et al., Nat. Commun. 2019, 10, 3307; Zhou et al. Sci. Adv. 2016, 2, e1600102). Nevertheless, most of the hyperbranched pBAEs which have been shown to have excellent transfection efficiency were developed for DNA delivery using plasmid DNA. There is not much literature reporting on the development of hyperbranched pBAEs for efficient siRNA delivery and gene silencing.

The major difference between DNA and siRNA is that siRNA has smaller size and higher rigidity than most plasmid DNA used for DNA delivery (Hagerman, Annu. Rev. Biophys. Biomol. Struct. 1997, 26, 139-156; Kebbekus, Draper, & Hagerman, Biochem. 1995, 34, 4354-4357). As a result, there is decreased multivalency of electrostatic interactions between cationic polymers and siRNA, thus weakening the siRNA complexation ability. Meanwhile, the rigidity may affect the self-assembly process of cationic polymers and siRNA into nanoparticles (Kozielski, Tzeng, De Mendoza, & Green, ACS Nano 2014, 8, 3232-41). Therefore, it would be more difficult to develop a gene carrier for efficient siRNA delivery and gene silencing.

The present invention provides hyperbranched poly(β-amino esters) (pBAEs) polymers useful for delivery of small interfering RNA (siRNA) to cells. The polymers of formula (I) are linear structures. The polymers of formula (II) are hyperbranched structures. The aim of synthesizing polymers of formula (I) is to optimize their monomer composition for good siRNA complexation capability. The branching properties and degree of branching density are then introduced to synthesize polymers of formula (II) in order to further optimize the performance of gene vectors such as cytotoxicity and gene silencing efficiency.

WO2016/020474A1 describes branched poly(β-amino esters) with polymer backbone containing monomer composition of bisphenol A ethoxylate diacrylate, 4-amino-1-butanol and trimethylolpropane triacrylate which are useful for delivery of plasmid DNA to a broad spectrum of several cell types. Patent US20190125874A1 describes branched poly(β-amino esters) with polymer backbone containing monomer composition of bisphenol A glycerolate diacrylate, 3-morpholinopropylamine and N-methyl-1,3-diaminopropane, which are useful for delivery of mRNA to lung epithelial cells. The polymers of the present invention differ from these prior art polymers are that they are synthesized from different buildings blocks and the nucleic acid agent they carry. The present invention is optimized to have optimal monomer composition and branching density, that can efficiently deliver siRNA with low cytotoxicity and high gene silencing efficiency for RNA interference (RNAi) mediated gene silencing therapy. The siRNA delivery performance of the present invention has been verified in both cells that are easy to transfect and cells that are difficult to transfect.

U.S. Pat. No. 8,562,966 B2 describes end modified poly((β-amino esters) useful for a variety of medical applications. The end modified poly((β-amino esters) are prepared by addition of nucleophilic reagent (example amine) to an acrylate terminated poly (β-amino esters). The resulting end modified polymers are useful in drug delivery, particularly in the delivery of polynucleotides.

US 2017/0216455 A1 describes biodegradable, hyperbranched poly (β-amino esters) for gene therapy such as nucleic acid transfection agents. The invention describes improved transfection efficiencies that are both safe and non-toxic. This invention utilizes tri-acrylate monomers which react directly via Michael addition with amine monomers to give hyperbranched poly (β-amino esters).

### BRIEF DESCRIPTION OF THE FIGURES

FIG. 1 depicts the synthesis and characterization of linear pBAEs. (1A) shows the amine monomers, diacrylate monomers and end-capping monomer used to synthesize linear pBAEs. (1B) shows a representative synthesis scheme. (1C) shows the characterization of linear pBAEs for siRNA complexation, studied by conducting agarose gel electrophoresis of the complexes at various weight ratios of polymer:siRNA. The arrows indicate the weight ratios where the siRNA mobility was completely retarded. A1B3, A2B2 and A2B3 are selected to synthesize their corresponding hyperbranched pBAEs due to their slightly better siRNA complexation ability.

FIG. 2 depicts the synthesis and characterization of hyperbranched pBAEs. (2A) shows the amine monomers, diacrylate monomers, end-capping monomer, and branching monomer used to synthesize hyperbranched pBAEs. (2B) shows a representative synthesis scheme.

FIG. 3 depicts the characterization of linear and hyperbranched pBAEs for siRNA complexation, studied by conducting agarose gel electrophoresis of the complexes at various weight ratios of polymer:siRNA. The arrows indicate the weight ratios where the siRNA mobility was completely retarded. The incorporation of highly branched structure into the polymer backbone greatly enhances their electrostatic interaction with siRNA, resulting in better siRNA complexation ability.

FIG. 4 depicts the in vitro cytotoxicity of linear and hyperbranched pBAEs. (4A-C) shows the cell viability of 3T3 fibroblast cells determined after incubating with A2B2 series, A2B3 series and A1B3 series for 24 h (n=3, ±S.D). (4D-F) shows the cell viability of HeLa cells determined after incubating with A2B2 series, A2B3 series and A1B3 series for 24 h (n=3, ±S.D). Among all hyperbranched pBAEs, h(A2B3)-1 exhibits best biocompatibility with lowest cytotoxicity in both 3T3 cells (IC₅₀>320 μg/mL) and HeLa cells (IC₅₀=180 μg/mL).

FIG. 5 depicts the cellular uptake efficiency of the complexes formed by polymer/fluorescence-labelled siRNA. Lipofectamine 2000, Lipo2k, is used as positive control. The mean fluorescence intensity of HeLa cells is quantified by flow cytometry after transfected with Lipo2k and polymer for 24 h (n=3, ±S.D). h(A2B3)-1 shows the highest cellular uptake efficiency amongst the hyperbranched polymers, which is almost comparable with Lipo2k at a weight ratio of 30.

FIG. 6 depicts the formation of complexes and biophysical characterization of h(A2B3)-1/siRNA complexes. (6A) shows the chemical structure of top-performing hyperbranched pBAEs, h(A2B3)-1. (6B) illustrates the formation of h(A2B3)-1/siRNA complexes via electrostatic complexation. (6C-D) shows that particle size and zeta potential of h(A2B3)-1/siRNA complexes at various weight ratios (polymer:siRNA) (n=3, ±S.D). h(A2B3)-1 can complex the siRNA and form nanoparticles with size between 100-150 nm and positive surface charges.

FIG. 7 depicts the comparison of in vitro cytotoxicity and gene silencing efficiency between h(A2B3)-1 and commercially available Lipo2k. (7A-B) shows the cell viabilities of h(A2B3)-1 and h(A2B3)-1/siRNA complexes in HeLa and 3T3 cells, as compared to Lipo2k. h(A2B3)-1 exhibits better cytocompatibility and much lesser cytotoxicity than Lipo2k. (7C) shows the in vitro GFP silencing efficiency of Lipo2k and h(A2B3)-1 in GFP-expressing HeLa cell, assessed by measuring the cell population percentage of GFP-positive cells (n=3, ±S.D). (7D) shows the confocal images of GFP-expressing HeLa cell transfected with Lipo2k/siRNA and h(A2B3)-1/siRNA. h(A2B3)-1-mediated delivery of GFP siRNA results in significant reduction on GFP-positive cell population and achieves higher GFP silencing efficiency than Lipo2k at weight ratio of 30, 40 and 50.

FIG. 8 depicts the gene uptake and silencing of h(A2B3)-1 in hard-to-transfect primary neural cells, with commercial TKO as positive control. (8A-B) shows the confocal images of cortical neurons after transfection with naked Cy5-labeled oligonucleotides (Cy5-ODN) and h(A2B3)-Cy5-ODN. Scale bar: 20 μm. (8C) shows the enlarged local view with orthogonal views of the area marked in the merged image of 8B, which shows h(A2B3)-Cy5-ODN particles are uptaken by cortical neurons and aggregate near the cell nuclei. (8D) shows the gene knockdown efficiency of h(A2B3)-siGAPDH and TKO-siGAPDH in cortical neurons and oligodendrocyte progenitor cells (OPCs) (n=3, ±S.D). h(A2B3)-1 achieves significant gene knockdown up to ^{˜}34.8% in cortical neurons and ^{˜}53.4% in OPCs.

FIG. 9 depicts the polymer degradation at pH7.4 and pH5.0, and post-degradation cytotoxicity. (9A-B) siRNA complexation ability of h(A2B3)-1 after incubated at pH7.4 and pH5.0, assessed by agarose gel electrophoresis. (9C-D) shows the amount of free siRNA quantified using ImageJ in gel lanes of different weight ratios. The polymers degraded at pH7.4 and pH5.0, weakening their siRNA complexation ability and release the siRNA. (9E-F) shows the post-degradation cytotoxicity of h(A2B3)-1 against HeLa cells (n=3, ±S.D). The degraded h(A2B3)-1 is highly cyto-compatible and non-toxic.

FIG. 10 depicts a synthesis scheme of bioreducible h(A2B3)-1. The bioreducible disulfide monomers (SS) are incorporated into the h(A2B3)-1 polymer backbone to further improve the biocompatibility.

FIG. 11 depicts the incorporation of disulfide bond into h(A2B3)-1 polymer backbone enhanced the biocompatibility while maintaining the gene transfection efficiency. (A) Cell viability of HeLa cells determined after incubating with Lipo2k, h(A2B3)-1, and bioreducible h(A2B3)-1. (B) in vitro GFP silencing efficiency of Lipo2k, h(A2B3)-1, and bioreducible h(A2B3)-1 in GFP-expressing HeLa cell. Data represent mean±S.D. (n=3).

FIG. 12 depicts agarose gel electrophoresis retardation of the bioreducible h(A2B3)-1, S1 to S5, at various weight ratios. The arrows indicate the weight ratios where the siRNA mobility was completely retarded.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of acceptable salts, for example pharmaceutically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

The term “alkyl” refers to a radical of a straight-chain or branched hydrocarbon group having a specified range of carbon atoms (e.g., a “C₁₋₁₆ alkyl” can have from 1 to 16 carbon atoms). An alkyl group can be a saturated alkyl group or an unsaturated alkyl group, i.e., an alkyl group having one or more carbon-carbon double/triple bonds, i.e., an alkenyl or alkynyl group. Unless specified to the contrary, an “alkyl” group includes both saturated alkyl groups and unsaturated alkyl groups.

The term “aliphatic” as used herein refers to an organic system composed of one or more alkyl, heteroalkyl, cycloalkyl, heterocycloalkyl, aryl, and heteroaryl groups in any permissible configuration. An aliphatic group may include any permissible number of substituents or degrees of unsaturation. An aliphatic group may be defined by the number of carbon atoms present, but unless specified to the contrary may include any number of heteroatoms as well. By way of example, a C₂aliphatic group includes —CH₂CH₂—, —CH₂OCH₂—, —CH₂C(═O)—, oxiranyl, and —CF₂CF₂—.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. By way of example, a heteroC₁₋₆alkyl (which may also be designated a C₁₋₆heteroalkyl) group includes, but is not limited to, the following structures:

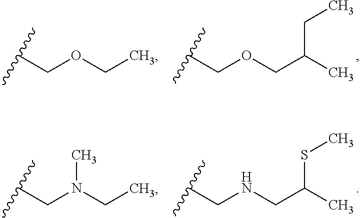

The term “heteroalkyl” preceded by a separate heteroatom refers to a heteroalkyl group bonded through the specified heteroatom. By way of example, a OC₁₋₆heteroalkyl group includes, but it not limited to, the following structures:

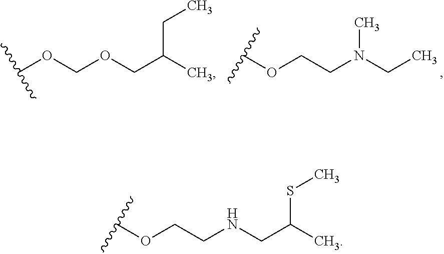

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Affixing the suffix “-ene” to a group indicates the group is a polyvalent moiety, e.g., boned to two or more groups. Alkylene is the polyvalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl (each of which parent groups as defined herein).

The term “alkoxy” refers to an alkyl group, as defined herein, appended to the parent molecular moiety through an oxygen atom.

The term “aryl” refers to a radical of a monocyclic or polycyclic (e.g., bicyclic or tricyclic) 4n+2 aromatic ring system (e.g., having 6, 10, or 14 T electrons shared in a cyclic array) having 6-14 ring carbon atoms and zero heteroatoms provided in the aromatic ring system (“C₆₋₁₄ aryl”). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 2H,6H′ 1,5,2-dithiazinyl, dihydrofuro[2,3 b]tetrahydrofuran, furanyl, furazanyl, imidazolidinyl, imidazolinyl, imidazolyl, IH-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

Unless specified to the contrary, the alkyl, alkenyl, alkynyl, heteroalkyl, heteroalkenyl, heteroalkynyl, carbocyclyl, heterocyclyl, aryl, and heteroaryl groups defined herein (and the “ene” versions of said groups) may be substituted or unsubstituted. A substituted group includes a non-hydrogen substituent at a position where in the unsubstituted version a hydrogen atom would be found. Substituents include, but are not limited to, halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NR^{a}R^{b}, —NR^{a}C(═O)R^{b}, —NR^{a}C(═O)NR^{a}NR^{b}, —NR^{a}C(═O)OR^{b}, —NR^{a}SO₂R^{b}, —C(═O)R^{a}, —C(═O)OR^{a}, —C(═O)NR^{a}R^{b}, —OC(═O)NR^{a}R^{b}, —OR^{a}, —SR^{a}, —SOR^{a}, —S(═O)₂R^{a}, —OS(═O)₂R^{a} and —S(═O)₂OR^{a}. R^{a} and R^{b} in this context can be the same or different and independently hydrogen, halogen, hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

As used herein, the designation of a polyvalent moiety without specifying the specific order of attachment is intended to cover all possible arrangements. By way of example, a compound represented by the formula:

A-X—B,

wherein X is NHC(═O) embraces both:

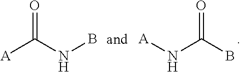

As used herein, a chemical bond depicted [symbol] represents either a single, double, or triple bond, valency permitting. By way of example,

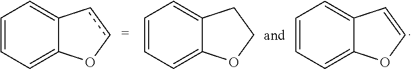

Some compounds disclosed herein may exist as one or more tautomers. Tautomers are interconvertible structural isomers that differ in the position of one or more protons or other labile atom. By way of example:

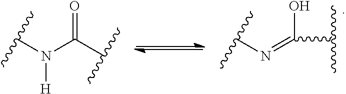

Unless stated to the contrary, a substituent drawn without explicitly specifying the point of attachment indicates that the substituent may be attached at any possible atom. For example, in a benzofuran depicted:

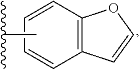

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃. A group having the subscript ‘0’ is understood to represent a null group as well. By way of example, in the compound CH₃—(X)_{z}—CH₃, if X is CH₂ and z is 0, then the compound has the formula CH₃—CH₃.

A bracketed group with no numeric subscript should be understood to include any number of repeating groups:

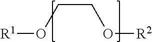

includes

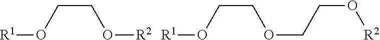

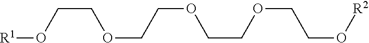

etc. . . .

A bracketed functional group with a subscripted variable or numeral should be understood to denote the number of repeated bracketed groups present. For example:

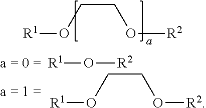

In certain instances, two or more variable groups may together form a ring. It is understood that any depicted atoms separated the identified groups will themselves form part of the ring:

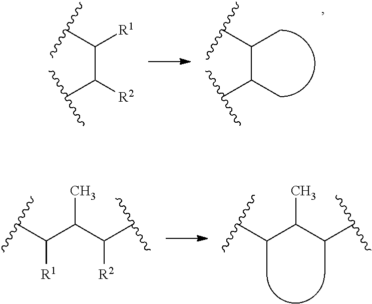

When the variable groups are substituted on an aromatic system the new ring will be a fused ring, and unless specified to the contrary may be either aromatic or non-aromatic, carbocyclic or heterocyclic:

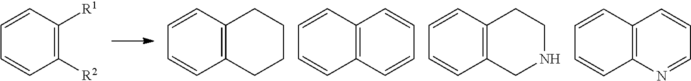

The ring may further be defined by the number of carbon atoms in the specific ring formed by the variable groups, which includes the atoms separating the variable groups:

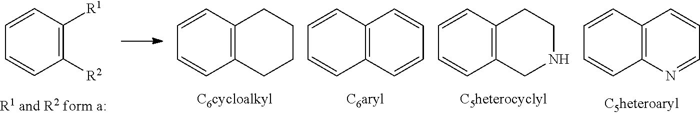

Each of the above results when R¹ and R² together form a six membered (or six atom) ring. Other rings, including 3, 4, 5, 7, and 8-member rings may also be formed, and may be further limited by a specified number of carbon atoms. Although the singular “a ring” may be used to define the group, unless specified to the contrary both monocyclic and polycyclic rings are possible:

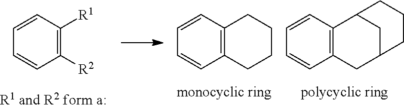

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or other labile atom. By way of example:

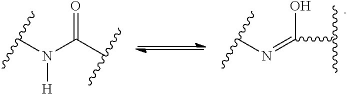

The prevalence of one tautomeric form over another will depend on the specific chemical compound as well as its local chemical environment. Unless specified to the contrary, the depiction of one tautomeric form is inclusive of all possible tautomeric forms.

Disclosed herein are polymer compositions. In some implementations, the polymer has the following structure:

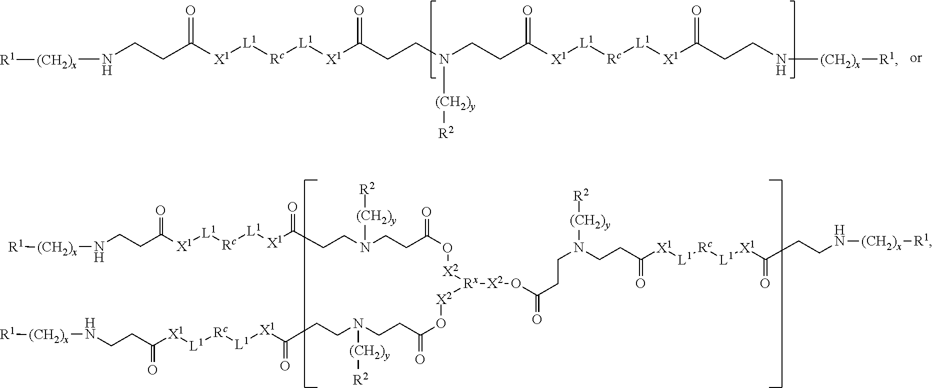

wherein:
R¹ is NH₂, NH(C₁₋₆alkyl), N(C₁₋₆alkyl)₂, C₁₋₈heterocyclyl, or C₁₋₈heteroaryl;
x is 1, 2, 3, 4, or 5;
X¹ is O or NH;
L¹ is null or a C₂₋₂₀aliphatic group;
R^{c} is a C₂₋₂₀aliphatic group;
R² is OH, NH₂, NH(C₁₋₆alkyl), N(C₁₋₆alkyl)₂, C₁₋₈heterocyclyl, or C₁₋₈heteroaryl;
y is 1, 2, 3, 4, or 5;
X² is null, CH₂ or CH₂CH₂; and
R^{x} is N or a C₁₋₆aliphatic group.

In certain implementations, R¹ and R² are not the same.

In certain implementations the polymer can have the formula:

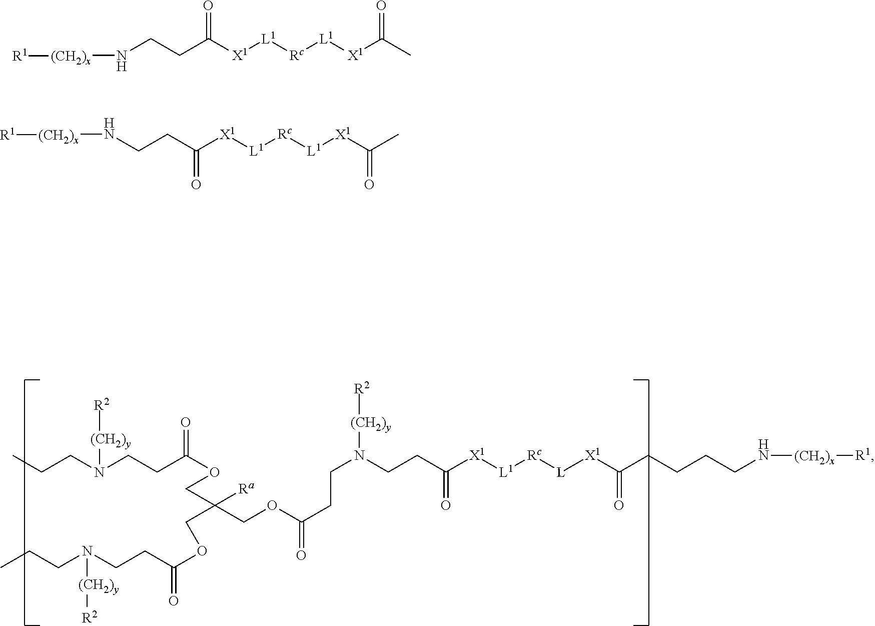

wherein R^{a} is H, CH₃, or CH₂CH₃.

In some implementations, R^{x} can be N, phenyl, pyridinyl, or C₁₋₄alkyl, for example a C₁alkyl having the formula: C—R^{a}, wherein R^{a} is H, CH₃, or CH₂CH₃.

In some implementations, R¹ is a C₄₋₈heterocyclyl group, optionally substituted one or more times by halo, hydroxy, COOH, NH₂, NH(C₁₋₅alkyl), N(C₁₋₅alkyl)₂, or O(C₁₋₅alkyl). In some implementations, R¹ is an unsubstituted C₄₋₈heterocyclyl group. In certain implementations, R¹ can include at least one secondary nitrogen atom (i.e., a nitrogen atom bearing two non-hydrogen substituents), present either in the C₄₋₈heterocyclyl group itself or as a substituent.

In various implementations, R¹ is a C₄₋₈heterocyclyl group having the formula:

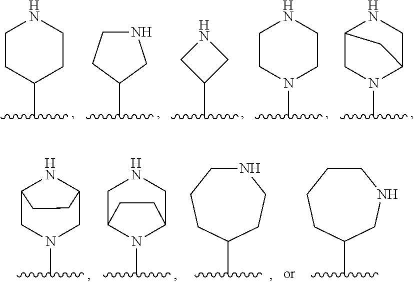

In certain implementations, x is 2, 3, or 4, preferably 2. In some implementations, x is 2 and R¹ is:

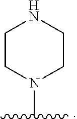 [SMILES: *N1CCNCC1 ; "."]

In certain implementations, X¹ is O.

L¹ can be any suitable divalent linking group, for example a C₂₋₁₀aliphatic group. In certain implementations L¹ can be a C₂₋₈alkylene group or C₂₋₈heteroalkylene group. In some implementations L¹ can be an unsubstituted C₂₋₈alkylene group or an unsubstituted C₂₋₈heteroalkylene group. In other implementations, L¹ can be a C₂₋₈alkylene group or C₂₋₈heteroalkylene group, substituted one or more times by halo, hydroxy, COOH, NH₂, NH(C₁₋₅alkyl), N(C₁₋₅alkyl)₂, or O(C₁₋₅alkyl). In some implementations L¹ can have the formula:

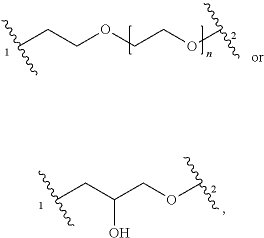

wherein n is 0-8, preferably 0, 1, or 2, and wavy line 1 represents the point of connection to X¹ and wavy line 2 represents the point of connection to R^{c}.

In certain implementations, some or all of the R^{c} groups can include a bioerodible moiety, Bioerodible moieties include disulfides, phosphonates, acetals, thioketals [e.g., R—S—CH₂—S—R, R—S—C(CH₃)₂—S—R], oxalate esters, boronic esters, and Schiff bases/imines. In certain implementations, some, but not all, of the R^{c} units include a bioerodible moiety, preferably a disulfide. In certain implementations, from 1-50%, from 1-25%, from 1-15%, from 1-10%, from 1-5%, from 1-2.5%, from 2.5-5%, from 5-10%, from 10-25%, from 15-30%, from 20-40%, or from 30-50% of the R^{c} groups include a bioerodible moiety.

In certain implementations, R^{c} is independently chosen from:

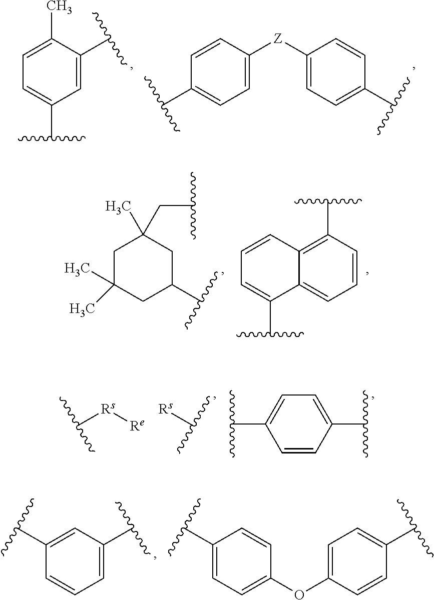

wherein Z is null, CH₂, C(CH₃)₂, O, or NH, R^{e} is a bioerodible group, for example S—S, S—CH₂—S, S—C(CH₃)₂—S, or O—C(═O)—C(═O)—O, and R^{s} is a C₂₋₆aliphatic group. In some implementations R^{s} is in each case CH₂CH₂, C(CH₃)₂CH₂, CH(CH₃)CH₂, CH₂CH₂CH₂, C(CH₃)₂CH₂CH₂, or CH(CH₃)CH₂CH₂, preferably R^{s} is in each case CH₂CH₂.

In some implementations, each instance of R^{c} in the polymer is the same moiety. In other implementations, the polymer may include multiple different R^{c} groups. For example, in some implementations R^{c} is independently:

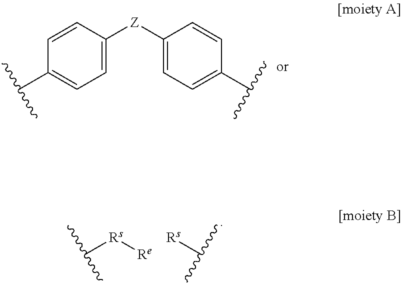

In some implementations, the ratio of [moiety A]:[moiety B] is from 1:0.01-0.5, from 1:0.01-0.25, from 1:0.01-0.1, from 1:0.1-0.2, from 1:0.2-0.3, from 1:0.3-0.4, from 1:0.4-0.5, from 1:0.1-0.25, or from 1:0.25-0.5. In certain implementations Z is CH₂ or C(CH₃)₂, or O.

In some implementations R^{c} is independently:

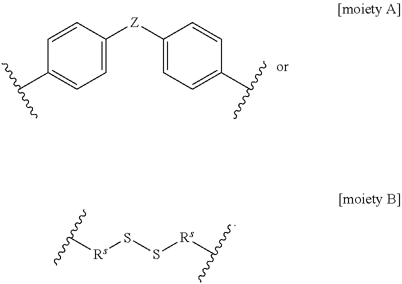

In some implementations, the ratio of [moiety A]:[moiety B] is from 1:0.01-0.5, from 1:0.01-0.25, from 1:0.01-0.1, from 1:0.1-0.2, from 1:0.2-0.3, from 1:0.3-0.4, from 1:0.4-0.5, from 1:0.1-0.25, or from 1:0.25-0.5. In certain implementations Z is CH₂.

In certain implementations, R² is H, OH, NH₂, NHCH₃, N(CH₃)₂, C₄₋₈heterocyclyl, or C₃₋₆heteroaryl. When R² is a C₄₋₈heterocyclyl or C₃₋₆heteroaryl, R² can be substituted one or more times by halo, hydroxy, COOH, NH₂, NH(C₁₋₅alkyl), N(C₁₋₅alkyl)₂, or O(C₁₋₅alkyl). In certain implementations, R² does not include any primary or secondary amine groups.

In some implementations R² is a C₄₋₈heterocyclyl or C₃₋₆heteroaryl having the formula:

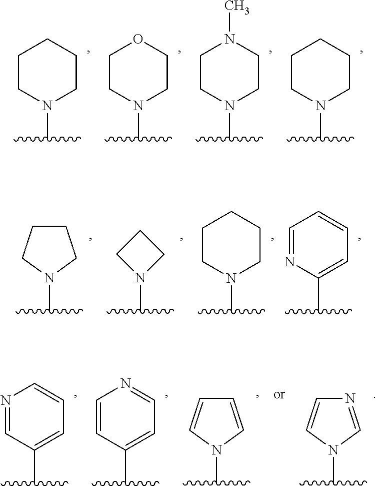

In some implementations y is 2, 3, or 4. In further implementations R¹ is OH or N-morpholinyl.

In certain implementations, R^{x} is N or a group having the formula:

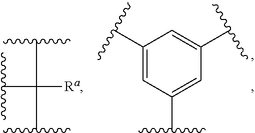

wherein R^{a} is H, CH₃, or CH₂CH₃.

In some implementations R^{x} is a group having the formula:

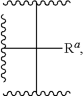

wherein R^{a} is CH₂CH₃ and X² is CH₂.

The disclosed polymers may be obtained by (a) synthesizing an uncapped polymer by a providing a mixture including a compound of Formula (1):

R²—(CH₂)_{y}—NH₂ [Formula (1)], and

a compound of Formula (2)

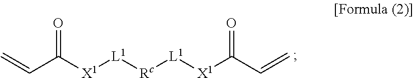

and
(b) reacting the uncapped polymer with a compound of Formula (4):

R¹—(CH₂)ₓ—NH₂ [Formula (4)],
wherein:
R¹ is NH₂, NH(C₁₋₆alkyl), N(C₁₋₆alkyl)₂, C₁₋₈heterocyclyl, or C₁₋₈heteroaryl;
x is 1, 2, 3, 4, or 5;
X¹ is O or NH;
L¹ is null or a linking group;
R^{c} is a C₂₋₂₀aliphatic group;
R² is OH, NH₂, NH(C₁₋₆alkyl), N(C₁₋₆alkyl)₂, C₁₋₈heterocyclyl, or C₁₋₈heteroaryl; and
y is 1, 2, 3, 4, or 5.

The uncapped polymer can have the following formula:

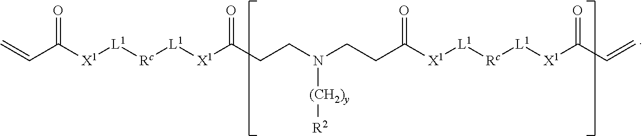

In certain implementations of the compound of Formula (1) R² is H, OH, NH₂, NHCH₃, N(CH₃)₂, C₄₋₈heterocyclyl, or C₃₋₆heteroaryl. When R² is a C₄₋₈heterocyclyl or C₃₋₆heteroaryl, R² can be substituted one or more times by halo, hydroxy, COOH, NH₂, NH(C₁₋₅alkyl), N(C₁₋₅alkyl)₂, or O(C₁₋₅alkyl). In certain implementations, R² does not include any primary or secondary amine groups.

In some implementations of the compound of Formula (1) R² is a C₄₋₈heterocyclyl or C₃₋₆heteroaryl having the formula:

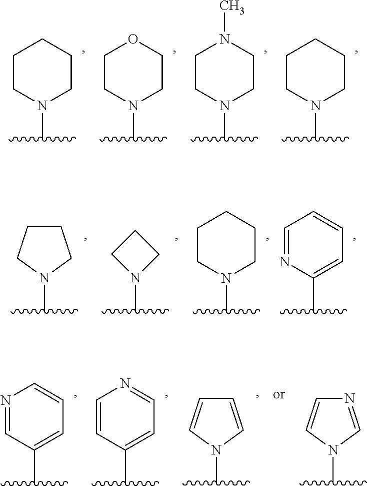

In some implementations of the compound of Formula (1), y is 2, 3, or 4. In further implementations of the compound of Formula (1), R¹ is OH or N-morpholinyl.

In some implementations of the compound of Formula (4), R¹ is a C₄₋₈heterocyclyl group, optionally substituted one or more times by halo, hydroxy, COOH, NH₂, NH(C₁₋₅alkyl), N(C₁₋₅ alkyl)₂, or O(C₁₋₅alkyl). In some implementations, R¹ is an unsubstituted C₄₋₈heterocyclyl group. In certain implementations, R¹ can include at least one secondary nitrogen atom (i.e., a nitrogen atom bearing two non-hydrogen substituents), present either in the C₄₋₈heterocyclyl group itself or as a substituent. In certain implementations, x is 2 or 3, preferably 2.

In some implementations of the compound of Formula (4), R¹ has the formula:

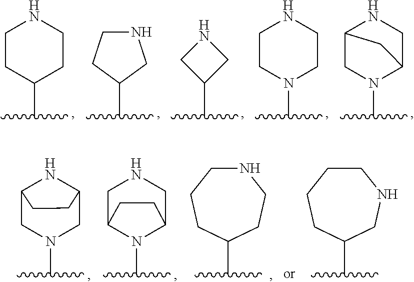

In some implementations of the compound of Formula (4), R¹ is:

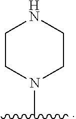 [SMILES: *N1CCNCC1 ; "."]

and x is 2.

In some implementations of the compound of Formula (2) X¹ is O, and L¹ can be any suitable divalent linking group, for example a C₂₋₁₀aliphatic group. In certain implementations L¹ can be a C₂₋₈alkylene group or C₂₋₈heteroalkylene group. In some implementations L¹ can be an unsubstituted C₂₋₈alkylene group or an unsubstituted C₂₋₈heteroalkylene group. In other implementations, L¹ can be a C₂₋₈alkylene group or C₂₋₈heteroalkylene group, substituted one or more times by halo, hydroxy, COOH, NH₂, NH(C₁₋₅alkyl), N(C₁₋₅alkyl)₂, or O(C₁₋₅alkyl). In some implementations, L¹ can have the formula:

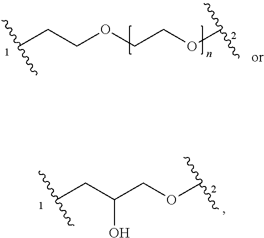

wherein n is 0-8, preferably 0, 1, or 2, and wavy line 1 represents the point of connection to X¹ and wavy line 2 represents the point of connection to R^{c}.

In some implementations of the compound of Formula (2) R^{c} is independently chosen from:

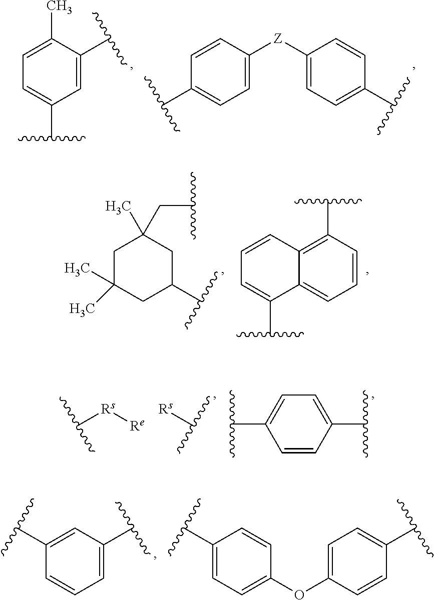

wherein Z:
is null, CH₂, C(CH₃)₂, O, or NH,
R^{e} is a bioerodible group, for example S—S, S—CH₂—S, S—C(CH₃)₂—S, or O—C(═O)—C(═O)—O, and
R^{s} is a C₂₋₆aliphatic group.

In some implementations R^{s} is in each case CH₂CH₂, C(CH₃)₂CH₂, CH(CH₃)CH₂, CH₂CH₂CH₂, C(CH₃)₂CH₂CH₂, or CH(CH₃)CH₂CH₂, preferably R^{s} is in each case CH₂CH₂.

In certain preferred implementations of the compound of Formula (2), R^{c} has the formula:

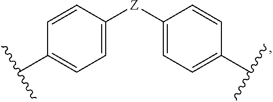

wherein Z is null, CH₂, C(CH₃)₂, O, or NH, preferably CH₂.

In certain implementations, the compound of Formula (2) is a compound of Formula (2a):

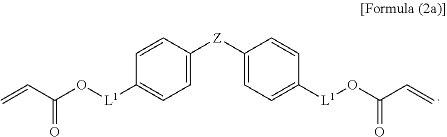

In certain implementations of the compound of Formula (2a), L¹ can be a C₂₋₈alkylene group or C₂₋₈heteroalkylene group. In some implementations L¹ can be an unsubstituted C₂₋₈alkylene group or an unsubstituted C₂₋₈heteroalkylene group. In other implementations, L¹ can be a C₂₋₈alkylene group or C₂₋₈heteroalkylene group, substituted one or more times by halo, hydroxy, COOH, NH₂, NH(C₁₋₅alkyl), N(C₁₋₅alkyl)₂, or O(C₁₋₅alkyl). In some implementations, L¹ can have the formula:

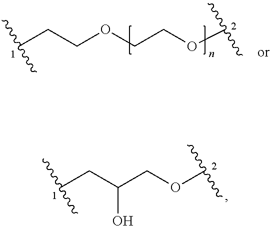

wherein n is 0-8, preferably 0, 1, or 2, and wavy line 1 represents the point of connection to the acrylate group and wavy line 2 represents the point of connection to the phenyl ring.

In certain implementations, the mixture can further include a crosslinking compound. In some implementations, the crosslinking compound is a tri-acrylate or a tetra-acrylate. In some implementations, the crosslinker can be a compound of Formula (3):

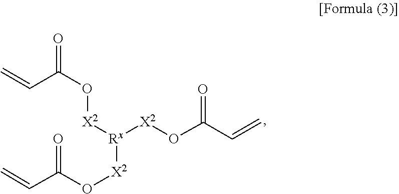

wherein X² is in each case independently null, CH₂ or CH₂CH₂; and
R^{x} is N or a C₁₋₆aliphatic group; when R^{x} is N, is it preferred that X² is CH₂CH₂.

In some implementations, R^{x} is phenyl (for example, the X² groups may be attached at the 1, 3, and 5 positions), pyridinyl, or C—R^{a}, wherein R^{a} is H, CH₃, or CH₂CH₃.

In some implementations, the compound of Formula (3) is selected from:

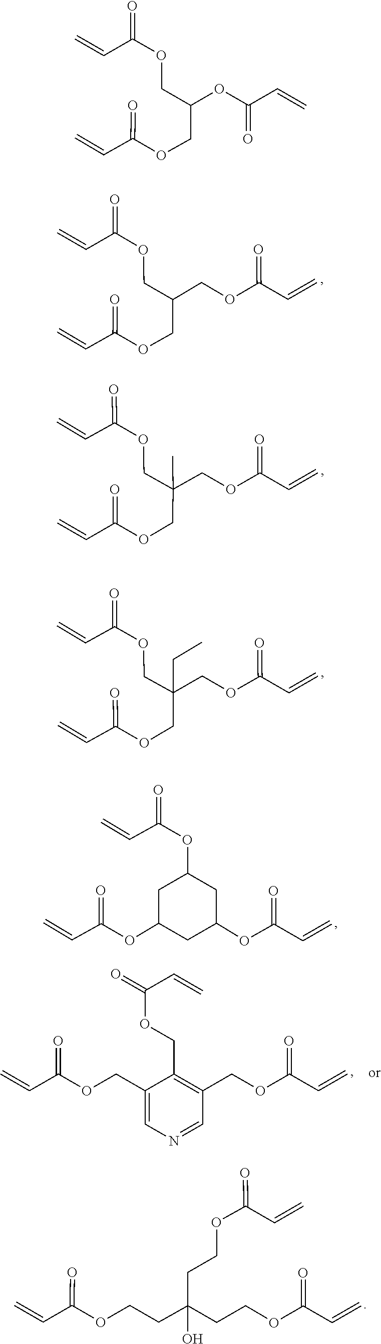

In some implementations, the mixture further includes a bioerodible compound. Bioerodible compounds include disulfides, phosphonates, acetals, thioketals [e.g., R—S—CH₂—S—R, R—S—C(CH₃)₂—S—R], oxalate esters, boronic esters, and Schiff bases/imines. In some implementations the bioerodible compound can be a compound of Formula (5):

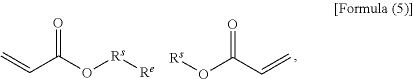

wherein R^{s} is a C₂₋₆aliphatic group and R^{e} is a bioerodible group. In some implementations R^{e} is S—S, S—CH₂—S, S—C(CH₃)₂—S—, or O—C(═O)—C(═O)—O. In some implementations, the bioerodible compound is a compound of Formula (5a):

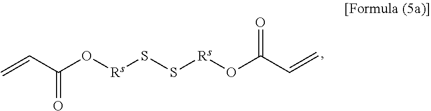

In some implementations, R^{s} is in each case CH₂CH₂, C(CH₃)₂CH₂, CH(CH₃)CH₂, CH₂CH₂CH₂, C(CH₃)₂CH₂CH₂, or CH(CH₃)CH₂CH₂, preferably R^{s} is in each case CH₂CH₂.

The mixture may further include a solvent, for example a polar aprotic solvent, a polar protic solvent, an apolar solvent, or a combination thereof. Exemplary solvents include DMSO, acetone, DMF, water, methanol, ethanol, ethyl acetate, and combinations thereof.

In certain implementations, the compound of Formula (1) and Formula (2) are present in the mixture in a molar ratio from 1.00-2.00:1, from 1.00-1.5:1, from 1.50-2.00:1, from 1.50-1.75:1, from 1.75-2.00:1, from 1.00-1.75:1, from 1.00-1.25:1, from 1.00-1.10:1, from 1.05-1.25:1, from 1.05-1.15:1, from 1.10-1.25:1, from 1.15-1.30:1, from 1.20-1.35:1, from 1.25-1.40:1, from 1.30-1.45:1, from 1.35-1.50:1, from 1.40-1.55:1, from 1.45-1.60:1, or from 1.50-1.65:1, preferably from 1.50-1.75:1.

In certain implementations, the compound of Formula (2) and Formula (3) are present in the mixture in a molar ratio from 1:0.1-0.75, from 1:0.5-0.75, from 1:0.25-0.75, from 1:0.25-0.50, or from 1:0.1-0.25.

In certain implementations, the compound of Formula (2) and Formula (5) are present in the mixture in a molar ratio from 1:0.01-0.50, from 1:0.01-0.35, from 1:0.01-0.2, from 1:0.01-0.1, from 1:0.1-0.50, from 1:0.1-0.35, from 1:0.1-0.2, from 1:0.2-0.3, from 1:0.3-0.4, or from 1:0.4-0.5.

In some implementations, the compound of Formula (4) may be employed in a suitable amount to ensure complete capping of the terminal acrylate groups in the uncapped polymer.

Also disclosed herein are compositions including the polymer and a nucleic acid. The polymer and nucleic acid may be present in a weight ratio (polymer:nucleic acid) from 5-500:1, from 5-250:1, from 5-100:1, from 5-50:1, from 5-25:1, from 10-100:1, from 10-50:1, from 10-25:1, from 20-40:1, from 25-50:1, from 25-75:1, from 40:60:1, from 50-100:1, from 50-75:1, or from 75-100:1.

The polymer/nucleic acid compositions may be provided as nanoparticle aggregates of the polymer and nucleic acid. In some implementations the nanoparticle can have a particle size (determined by DLS) from 10-1,000 nm, from 10-500 nm, from 10-250 nm, from 10-100 nm, from 10-50 nm, from 10-25 nm, from 25-100 nm, from 25-250 nm, from 50-100 nm, from 50-150 nm, from 50-250 nm, from 100-200 nm, from 100-250 nm, from 250-500 nm, or from 500-1,000 nm.

In some implementations, the nanoparticles can be characterized by Zeta potential. In certain implementations, the nanoparticles can have a Zeta potential (determined by DLS) from 2-100 mV, from 2-50 mV, from 2-25 mV, from 2-10 mV, from 5-25 mV, from 5-15 mV, from 10-25 mV, from 15-30 mV, from 25-50 mV, from 25-75 mV, from 50-75 mV, or from 75-100 mV.

A variety of different nucleic acids may be combined with the disclosed polymers. In implementations the nucleic acid can be long noncoding (Inc) ribonucleic acid (RNA) (lncRNA), small non-coding (snc) RNA (sncRNA), Piwi-interacting RNA (piRNA), interfering RNA (RNAi), small interfering RNA (siRNA), repeat associated small interfering RNA (rasiRNA), microRNA (miRNA), and/or antisense oligonucleotide. In some implementations the polymer may be combined with a two or more different types of nucleic acids.

The disclosed compositions may be made by mixing the polymer (or combinations of polymers) and a nucleic acid. The polymer and nucleic acid aggregate due to electrostatic interactions. The mixing may take place in an aqueous solvent, for example an aqueous buffer having a pH from 6-8, 6-7, 6.5-7, 6.5-7.5, 7-7.5, 7-8, or 7.5-8.

Also disclosed herein are methods of administering nucleic acids to a subject in need thereof using the disclosed compositions. Also disclosed herein are methods of providing gene therapy to a subject in need thereof using the disclosed compositions. Also disclosed herein are methods of silencing genes to a subject in need thereof using the disclosed compositions.

The compositions may be administered enterally, parenterally, intranasally, vaginally, by inhalation, or a combination thereof. When the composition is administered enterally, it may be administered by oral administration, sublingual administration, buccal administration, rectal administration, or a combination thereof. When the composition is administered parenterally, it may be administered by intramuscular injection, intravenous injection, subcutaneous injection, or a combination thereof.

In certain implementations, the composition is an aqeuous composition that may be administered by intravenous injection.

The compositions may be used to treat a variety of disease states in subjects. In some implementations, the compositions can be used to treat infectious disease in a subject in need thereof. Exemplary infectious diseases include viral infections, for example HIV, hepatitis A, hepatitis B, hepatitis C, HPV, or West Nile virus.

In some implementations, the compositions can be used to treat cancer in a subject in need thereof. In some implementations the compositions can be used to treat leukemia.

In some implementations, the compositions can be used to treat respiratory disease in a subject in need thereof. In some implementations the compositions can be used to treat asthma, COPD, or cystic fibrosis.

In some implementations, the compositions can be used to treat neurodegenerative disorders in a subject in need thereof. In some implementations the compositions can be used to treat ALS or Huntington's disease.

### EXAMPLES

The following examples are for the purpose of illustration of the invention only and are not intended to limit the scope of the present invention in any manner whatsoever.

Trimethylolpropane triacrylate (TMPTA), bisphenol A ethoxylate diacrylate, bisphenol A glycerolate (1 glycerol/phenol) diacrylate, 4-(2-aminoethyl) morpholine, 4-amino-1-butanol, 1-(2-aminoethyl)piperazine, acryloyl chloride, triethylamine (TEA), bis(2-hydroxylethyl) disulfide, anhydrous dimethyl sulfoxide (DMSO) and anhydrous tetrahydrofuran (THF) were purchased from Sigma-Aldrich. Lipofectamine 2000 (Lipo2k), Alexa Fluor® Red siRNA, Silencer™ GFP siRNA and negative control siRNA were purchased from Life Technologies for transfection studies.

DNase 1 type IV, poly-D-lysine (PDL), Bovine Serum Albumin (BSA, A9647-100G), N-acetyl cysteine NAC Sigma A8199, D-biotin Sigma B4639 ITS solution (Sigma 13146), all the components of SATO (Putrescine Sigma P-5780, L-Thyroxine Sigma T-1775, Tri-iodothyroxine Sigma T-6397, Progesterone Sigma P-8783, Bovine Serum Albumin fraction V Sigma A-4919), Cytarabine (PHR1787), and Triton X-100 were purchased from Sigma-Aldrich. Mounting Medium (SP15-500) was purchased from Fisher Scientific. Alexa-Fluor Goat anti-Rabbit 488, Alexa-Fluor Donkey anti-Rat 555, Alexa-Fluor Donkey anti-Mouse 555, DAPI (4′,6-diamidino-2-phenylindole), minimum essential media (MEM), DMEM high glucose, neurobasal medium (NB), fetal bovine serum (FBS, 10270106), Glutamax (35050061), B-27 supplement (17504044), N2 supplement (17502048), phosphate buffered saline (PBS, 10010023), Dulbecco's phosphate-buffered saline (DPBS, 14040133) and penicillin/streptomycin (Pen/Strep) were purchased from Life Technologies. Rat anti-MBP (aa82-87) was purchased from Bio-Rad. Mouse anti-Tuj1 (#MMS-435P) was purchased from BioLegend. Papain suspension was purchased from Worthington. 4% paraformaldehyde solution (sc-281692), 10× phosphate buffered saline (PBS) were purchased from Axil Scientific. RNA extraction kit was purchased from Qiagen (Singapore). Reversal transcript kit (NEB #E3010) and qPCR kit (NEB #M3003) were purchased from New England Biolabs Inc. TransIT-TKO (MIR2150) was purchased from MirusBio. All the primers and siRNA are pre-designed sequences that were purchased from Integrated DNA Technologies.

### Proton Nuclear Magnetic Resonance (1H NMR) Measurements

The polymers were dissolved in deuterated DMSO and characterized by 1H NMR. The proton NMR spectra were recorded on a Bruker Avance 400 MHz NMR spectrometer with at least 8 scans. Chemical shifts were quoted relative to the solvent peak (DMSO-d6 at δ 2.50 ppm).

### Agarose Gel Electrophoresis Assay

pBAE/siRNA complexes were prepared by mixing siRNA solution with polymer solution at varying polymer:siRNA w/w ratios. The solutions were vortexed and incubated at room temperature for 30 min before use. 20 μL of complexes solution at varying w/w ratios were mixed with 2 μL of 10× BlueJuice loading buffer. Next, the sample was loaded on 1% agarose gel containing SYBR gold gel stain. The measurement was conducted in 1× Tris-acetate-EDTA (TAE) buffer for 20 min under 100 V in a Sub-Cell system. siRNA bands were visualized under UV exposure using a G-box imaging system.

### Example 1: Synthesis and Characterization of Linear pBAEs

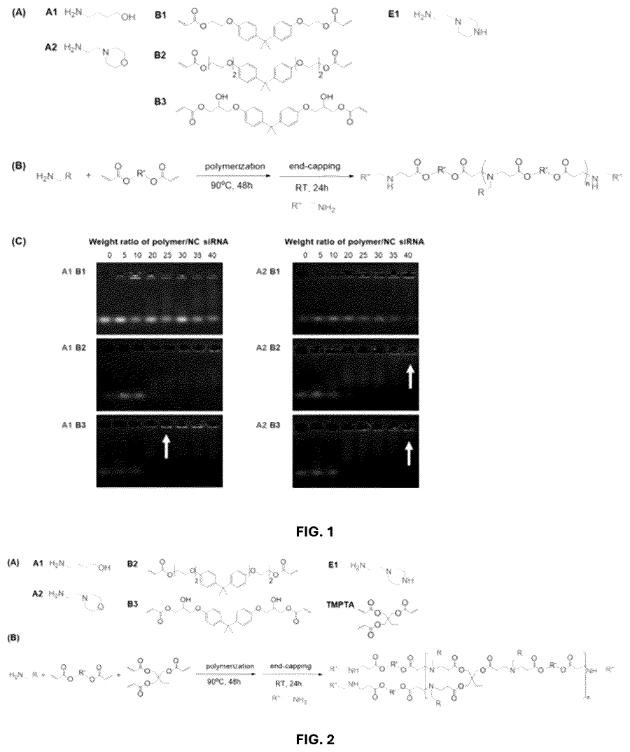

A subset of monomers depicted in FIG. 1A was used to optimize the monomer composition of linear PBAEs for efficient electrostatic complexation with siRNA. The linear pBAEs were synthesized via Michael addition reaction (FIG. 1B). To synthesize the linear pBAEs, amine monomers (A1 or A2) and diacrylate monomers (B1, B2 or B3) were first dissolved in DMSO (500 mg/ml) and reacted at 90° C. for 48 h. The [vinyl]: [NH] ratio of these reactions was set at 1.2:1. After 48 h reaction, the reaction solution was cooled to room temperature (RT) and diluted with DMSO at 100 mg/ml. End-capping amine monomer (E1) was added to end-cap the base polymer by a further 24 h reaction at room temperature. The final polymers were purified by precipitation in diethyl ether three times, vacuum dried overnight, and then stored at −20° C.

Six linear pBAEs using the monomers listed above, namely A1B1, A1B2, A1B3, A2B1, A2B2 and A2B3, were synthesized. 1H NMR was used to confirm the successful copolymerization of the monomers and chemical structure of the polymers. The siRNA binding efficiency of these six linear pBAEs was characterized using agarose gel electrophoresis assay to compare and identify the optimal monomer composition for efficient siRNA complexation (FIG. 1C).

Two linear pBAEs using B1 as diacrylate monomer showed poor siRNA condensation ability. Polymers using A2 as the amine monomer and B2 or B3 as diacrylate monomer resulted in slightly better siRNA complexation, which could stop siRNA migration at the polymer:siRNA weight ratio of 40:1. Among all, A1B3 gave the lowest weight ratios to fully retard the siRNA migration at w/w 25. Based on these gel retardation results, A1B3, A2B2 and A2B3 were selected to synthesize their corresponding hyperbranched pBAEs due to their slightly better siRNA complexation ability.

The disulfide monomers (SS) were firstly prepared according to the literature reported previously. Briefly, bis(2-hydroxylethyl) disulfide (2 g, 12.9 mmol) and TEA (9 mL, 64.8 mmol) were dissolved in 26 mL of anhydrous THF. Next, acryloyl chloride (6.287 mL, 77.8 mmol) dissolved in 4 mL of anhydrous THF was added dropwise into the mixture of the flask in an ice bath. The resulting heterogeneous mixture was stirred at 25° C. for 24 h under argon environment. After reaction, triethylamine hydrochloride precipitate was removed by filtration, and the solvent was removed by rotary evaporation. The product was dissolved in dichloromethane (DCM), washed three times with an aqueous solution of 0.1 M Na₂CO₃, two times with distilled water and once with brine. Finally, the product was dried using anhydrous MgSO₄ and obtained by removing the solvent under reduced pressure.

To synthesize bioreducible h(A2B3)-1 (Scheme 1), TMPTA, A2 (amine monomer), B3 (diacrylate monomer) and SS (disulfide monomers) were dissolved in DMSO (500 mg/ml) and reacted at 90° C. for 48 h. The [vinyl]: [NH] of these reactions were set at 1.2:1. After 48 h reaction, the mixture was cooled to RT and diluted to 100 mg/ml with DMSO. 1-(2-aminoethyl)piperazine was added to end-cap the base polymer and react at RT for 24 h. The final polymers were purified by precipitation in diethyl ether three times, vacuum dried overnight, and then stored at −20° C.

The bioreducible h(A2B3)-1 with different disulfide percentage were characterized by ¹H NMR. Their siRNA complexation ability was evaluated by gel electrophoresis assay. In vitro cytotoxicity was analyzed using MTT assay and siRNA transfection efficiency was studied according to the protocol above.

### Example 2: Synthesis and Characterization of Hyperbranched pBAEs

A subset of chosen monomers depicted in FIG. 2A was used to synthesize hyperbranched pBAEs. TMPTA was worked as branching monomer to obtain hyperbranched polymer structure. Three groups of monomer compositions were chosen to synthesize hyperbranched pBAEs, which were A2B2, A2B3, and A1B3. The hyperbranched pBAEs were synthesized via Michael addition reaction (FIG. 2B). To synthesize the hyperbranched pBAEs with these monomer compositions, TMPTA, amine monomers (A1 or A2) and diacrylate monomers (B2 or B3) with designed molar ratios were dissolved in DMSO (500 mg/ml) and reacted at 90° C. for 48 h. After 48 h reaction, reaction solution was cooled to room temperature and diluted with DMSO at 100 mg/ml. Monomer E1 was added to end-cap the base polymer and reacted for 24 h. The final polymers were purified by precipitation in diethyl ether three times, vacuum dried overnight, and then stored at −20° C.

To study the influence of branching density on gene delivery efficiency and cytotoxicity, two different ratios of TMPTA to diacrylate monomer were used to synthesize two hyperbranched pBAEs with different branching densities, namely as h(AB)-1 and h(AB)-2. The ratio of TMPTA to diacrylate for h(AB)-1 was 0.25:1 whereas the ratio of TMPTA to diacrylate for h(AB)-2 was 0.5:1. Generally, h(AB)-2 is more branched than h(AB)-1 due to its higher TMPTA molar ratio. The [vinyl]: [NH] ratio of these reactions was set at 1.2:1.

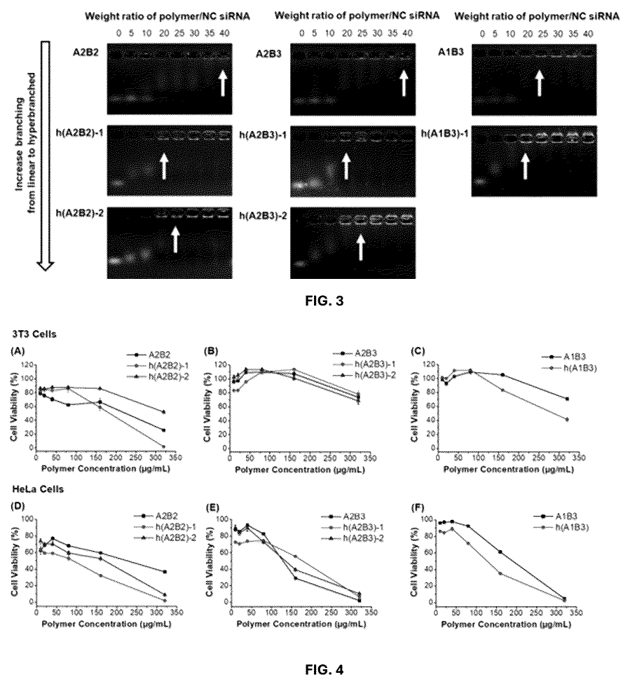

Five hyperbranched pBAEs using the monomers listed above and two branching densities, namely h(A2B2)-1, h(A2B2)-2, h(A2B3)-1, h(A2B3)-2, and h(A1B3)-1, were synthesized. The successful synthesis of hyperbranched pBAEs for the chosen monomer compositions were confirmed by measurement of the 1H NMR. Their siRNA complexation ability was also evaluated by gel electrophoresis assay (FIG. 3) to investigate the gene delivery properties of hyperbranched polymers as compared to their corresponding linear structure.

The gel retardation results demonstrated that all hyperbranched pBAEs could achieve lower weight ratio to completely retard siRNA mobility, as compared to their corresponding linear pBAEs. Linear polymer A2B2 stopped siRNA migration at the polymer:siRNA weight ratio (w/w) of 40. The introduction of hyperbranching structure enhance the siRNA complexation ability, which fully retard the siRNA migration at w/w 20 and 25 for h(A2B2)-1 and h(A2B2)-2, respectively. At w/w 40, the linear polymer A2B3 inhibited siRNA migration. However, by introducing a hyperbranching structure, the siRNA complexation ability improved greatly, leading to complete retardation of siRNA migration at w/w 20 and 25 for h(A2B3)-1 and h(A2B3)-2, respectively. In A1B3 series, the linear polymer A1B3 stopped siRNA migration at w/w 25. Nonetheless, with the incorporation of a hyperbranching structure, the siRNA complexation ability slightly increased, resulting in complete retardation of siRNA migration at w/w 20 for h(A1B3)-1.

These results demonstrated that incorporation of highly branched structure into the polymer backbone greatly enhances their electrostatic interaction with negatively charged siRNA, resulting in better siRNA complexation ability. This may be due to the increased cationic charge density of hyperbranched polymer as compared to their corresponding linear structure. Hyperbranched polymers have higher cationic charge density, hence providing larger capacity of electrostatic interaction with negative charged nucleic acid and subsequently enhancing the gene complexation ability.

### Example 3: Identification of Top Performing Hyperbranched pBAE for siRNA Delivery

HeLa cells (ATCC) and 3T3 cells were cultured in DMEM containing 10% fetal bovine serum (FBS), 1% antibiotic antimycotic solution (100×) at 37° C. in a humidified and 5% CO2 incubators.

HeLa cells and 3T3 cells were seeded in 96-well plates at a density of 1×10⁴ cells per well in 100 μL of DMEM complete medium. After 24 h incubation, the medium was removed and replaced by 100 μL of culture medium containing serial dilutions of polymers, continuing incubating the cells for 24 h. Thereafter, 50 μL of MTT solution (1 mg/ml) was added to each well and incubated for 2 h. Next, MTT solution was removed and 100 μL of isopropanol was added into each well. The absorbance of each well was measured by Tecan Spark microplate reader at 570 nm. The relative cell viability was calculated with the following equation: cell viability (%)=OD₅₇₀₍ₛₐₘₚₗₑ₎/OD_{570(untreated)}×100%. Data is means of triplicate measurements.

HeLa cells were seeded in 24-well plates at a density of 5×10⁴ cells per well and then incubated for 24 h before transfection. 50 μL of polymer/siRNA complexes containing 15 pmol of Alexa Fluor® Red siRNA (Invitrogen) was prepared at w/w ratios of 20 and 30. The culture medium was removed and 450 μL of serum-free medium (Welgene) was added into the wells. 50 μL of complexes solution were added to each well to obtain a final RNA concentration of 30 nM and incubated for 24 h. Control experiments were performed with Lipo2k, as instructed by the vendor's manual (Invitrogen). After 24 h of transfection, the medium was removed; the cells were harvested using trypsin solution, washed with PBS, and fixed in 4% parafomaldehyde. The samples were measured by BD LSRFortessa X-20 flow cytometry. Untreated cells were used as control for background calibration. Mean fluorescence intensity (MFI) of PE-positive cells was measured and used for transfection and uptake analysis.

The gene delivery capability of cationic polymers is dependent on molecular weight and branching of the polymer which directly affect their amino charge density (Fischer et al., Bioconjugate Chem. 2002, 13, 1124-1133). High molecular weight and branching of the polymer can enhance their gene transfection efficiency; however, they also increase the cytotoxicity due to the high density of amino groups and high molecular weight. The major concern which limits the application of cationic polymer in gene delivery is their toxicity both in vitro and in vivo (Pun & Davis, Bioconjugate Chem. 2002, 13, 630-9; Yhee et al., Bioconjugate Chem. 2013, 24, 1850-1860). Hence, polymer cytotoxicity has become an important consideration on selection of top-performing cationic polymers. To identify the top-performing hyperbranched pBAE polymer, the in vitro cytotoxicity of synthesized linear pBAEs and hyperbranched pBAEs was first evaluated in 3T3 fibroblasts and HeLa cells using MTT assay (FIG. 4). The table below summarizes the concentrations of the polymer that reduced viable cell number by 50%, also known as IC₅₀.

IC₅₀ (μg/mL)

| Polymers | 3T3 cells | HeLa cells |
|---|---|---|
| A2B2 | 225 | 230 |
| h(A2B2)-1 | 185 | 90 |
| h(A2B2)-2 | >320 | 170 |
| A2B3 | >320 | 130 |
| h(A2B3)-1 | >320 | 180 |
| h(A2B3)-2 | >320 | 135 |
| A1B3 | >320 | 190 |
| h(A1B3)-1 | 290 | 130 |

In A2B2 series, the incorporation of branching unit into the polymer backbone slightly increased the polymer toxicity of h(A2B2)-1. Although h(A2B2)-2 is more branched than h(A2B2)-1, the cytotoxicity is lower. In A2B3 series, the introduction of branching unit did not significantly affect the cytotoxicity. In 3T3 fibroblast cells, linear A2B3, hyperbranched h(A2B3)-1 and h(A2B3)-2 showed no cytotoxicity even at highest concentration. In HeLa cells, h(A2B3)-1 showed better biocompatibility with an IC50 value of 180 μg/mL as compared to A2B3 and h(A2B3)-1 polymer. Meanwhile, in A1B3 series, slightly increased polymer toxicity with reduced IC₅₀ was observed in the hyperbranched h(A1B3)-1 as compared to the linear A1B3. Among all hyperbranched pBAEs, h(A2B3)-1 exhibited best biocompatibility with lowest cytotoxicity in both 3T3 cells (IC₅₀>320 μg/mL) and HeLa cells (IC₅₀=180 μg/mL).

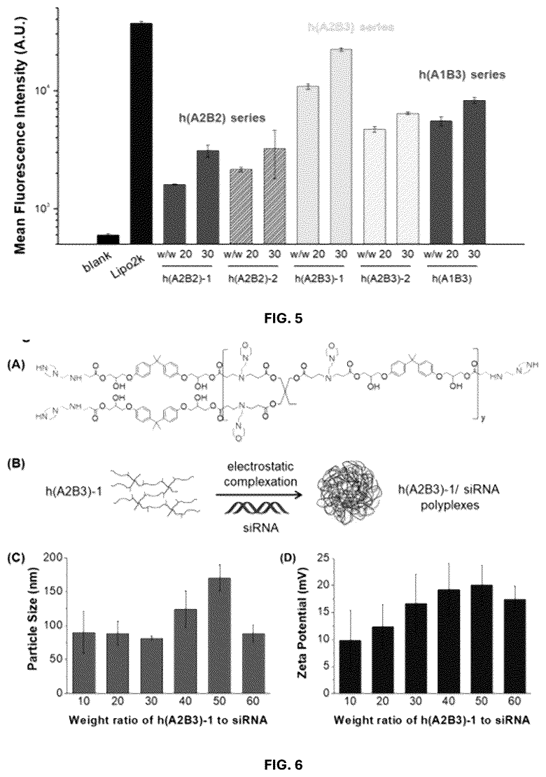

Cellular uptake of the gene carrier is a key factor to achieve high gene transfection efficiency. Therefore, besides cytotoxicity, the in vitro siRNA uptake efficiency of the synthesized hyperbranched pBAEs was evaluated in HeLa cells (FIG. 5). Commercial transfection agent Lipofectamine 2000 (Lipo2k) at vector: siRNA weight ratio of 5 was used as the positive control. HeLa cells were transfected with polymer/fluorescence-labelled siRNA for 24 h. The higher the cellular uptake efficiency of the gene carrier, the stronger the mean fluorescence intensity of the transfected cells.

h(A2B2) series showed the lowest uptake efficiency. On the other hand, the h(A1B3) and h(A2B3)-2 exhibited similar uptake efficiencies, which were slightly better than the h(A2B2) series. Meanwhile, h(A2B3)-1 showed the highest cellular uptake efficiency amongst the hyperbranched polymers, indicating that optimal branching degree in h(A2B3)-1 will promote better transfection capability than higher branching as in h(A2B3)-2. In particular, h(A2B3)-1 at a weight ratio of 30 showed the strongest mean fluorescence intensity, which was almost comparable with Lipo2k.

Based on the results above, h(A2B3)-1 with optimal monomer composition and branching degree was chosen to be the top-performing gene vector for siRNA delivery owing to its good biocompatibility, low cytotoxicity (with IC₅₀ value up to 180 μg/mL), and good cellular uptake efficiency which are comparable with Lipo2k.

### Example 4: Biophysical Properties of h(A2B3)-1/siRNA Complexes

The particle size and zeta potential of the complexes were determined via DLS using a Malvern Zetasizer NanoZS. The complexes solution at varying w/w ratio was first prepared and diluted to 1 mL with sodium acetate buffer. The samples were measured with DLS immediately after preparation to obtain the particle size, polydispersity index (PDI) and zeta potential. All measurements were done in triplicates.

The chemical structure of the optimal hyperbranched pBAEs, h(A2B3)-1, is shown in FIG. 6A. To study the biophysical properties of h(A2B3)-1/siRNA complexes, particle size and zeta potential of the complexes at different weight ratios of polymer:siRNA were measured. As shown in FIG. 6B-D, h(A2B3)-1 can complex the siRNA and form nanoparticle complexes with size between 100-150 nm and positive surface charges. The features of small particle size and positive surface charges could contribute to efficient cellular uptake which eventually enhances the gene transfection efficiency.

### Example 5: Comparison Study of h(A2B3)-1 with Lipo2k

Comparison of In Vitro Cytotoxicity Between h(A2B3)-1 and Lipo2k

Lipo2k and h(A2B3)-1 with increasing concentration were added into HeLa cells and 3T3 cells and then incubated for 24 h. h(A2B3)-1 complexes containing 3 pmol of siRNA at polymer:siRNA w/w ratio of 30 were prepared with increasing concentration. Lipo2k/siRNA complexes at w/w ratio of 5 with increasing concentration was used as the standard for toxicity comparison. The complexes solution was added into the cells and incubated for 24 h. The cell viability (%) was then determined by the MTT assay after 24 h incubation.

HeLa cells were plated at a seeding density of 8×10⁴ cells/well in 24-well plates and incubated for 18 h before pEGFP transfection. The cells were first transfected with Lipo2k/pEGFP complexes (1 μg of pEGFP plasmid DNA per well, using 1 μL Lipo2k) for 6 h in serum-free medium. Next, 50 μL of h(A2B3)-1/siRNA complexes containing 15 pmol of Silencer™ GFP siRNA (Invitrogen) or negative control siRNA (NC siRNA) was prepared at different weight ratios of 20, 30, 40 or 50. After transfection of pEGFP for 6 h, the medium was removed and replaced with 450 μL serum-free medium. 50 μL of complexes solution was added into the well to obtain a final RNA concentration of 30 nM and the cells were further incubated for 24 h. Control experiments were performed with Lipo2k, as instructed by the vendor's manual (Invitrogen). After 24 h of transfection, the medium was removed; the cells were harvested using trypsin solution, washed with PBS, and fixed in 4% parafomaldehyde. The samples were measured by BD LSRFortessa X-20 flow cytometry. Untreated cells were used as control for background calibration. Cell population percentage of FITC-positive cells was measured and used for transfection analysis.

The GFP silencing efficiency of h(A2B3)-1 was further investigated using confocal microscopy. The cells were transfected with complexes containing 15 pmol of Silencer™ GFP siRNA (Invitrogen) or NC siRNA for 24 h. After 24 h of transfection, the medium was removed and the cells were washed with PBS three times, fixed with 4% paraformaldehyde for 20 min and stained with DAPI solutions for 10 min. The cells were imaged under Zeiss LSM 800 confocal laser scanning microscope.

### Results and Discussion

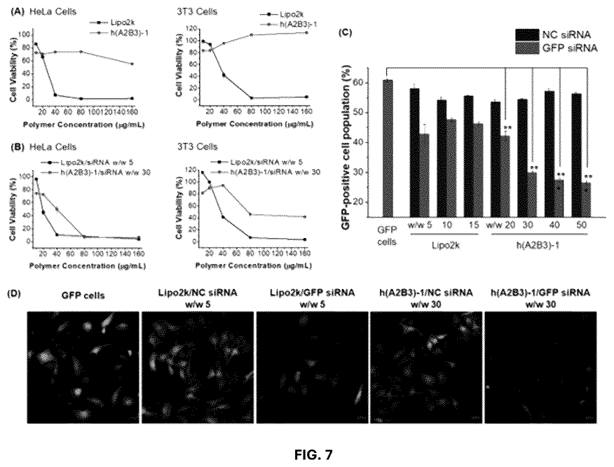

To demonstrate the good cytocompatibility and excellent siRNA transfection efficiency of h(A2B3)-1, in vitro cytotoxicity assay and Green Fluorescent Protein (GFP) silencing study were conducted with Lipo2k as positive control. As demonstrated in FIG. 7A, h(A2B3)-1 could maintain over 50% of cell viability even at the high polymer concentration up to 160 μg/mL whereas cell viability of 3T3 and HeLa cells treated with commercial Lipo2k dropped to 0% at 80 and 40 μg/mL, respectively. This indicated better cytocompatibility and much lesser cytotoxicity of h(A2B3)-1 as compared to Lipo2k. In the form of complexes (FIG. 7B), the h(A2B3)-1/siRNA showed some toxicity towards the cells at polymer concentration above 40 μg/mL, but it still exhibited lesser toxicity than Lipo2k/siRNA. The increased cytotoxicity might be because more polymers were taken up by the cells when polymer was complexed with siRNA and formed positively charged nanoparticles which facilitate the cellular uptake via electrostatic attraction. As the polymer concentration used for in vitro transfection study is between 0-20 μg/mL, the h(A2B3)-1/siRNA complexes did not cause any toxicity on the cells within this concentration range.

Next, the siRNA transfection efficiency of h(A2B3)-1 was investigated using GFP siRNA as positive control and NC siRNA (scramble non-coding siRNA) as negative control. Lipo2k/siRNA at weight ratio of 5, 10 and 15 were used as the gold standard. As shown in FIG. 7C, h(A2B3)-1-mediated delivery of GFP siRNA into HeLa cells resulted in significant reduction on GFP-positive cell population at all weight ratios (^{˜}19%-34% reduction) as compared to untreated cells. No GFP silencing effect was observed on cell transfection study with h(A2B3)-1/NC siRNA. On the other hand, the reduction of GFP cell population that was achieved by Lipo2k/GFP siRNA was only ^{˜}13%-18%. Compared with Lipo2k, h(A2B3)-1 exhibited comparable GFP silencing efficiency at weight ratio of 20, whereas it achieved higher GFP silencing efficiency at weight ratio of 30, 40 and 50. The confocal images (FIG. 7D) revealed that h(A2B3)-1/NC siRNA gave no silencing effect while h(A2B3)-1/GFP siRNA successfully silenced the GFP expression of the cells. By comparing the GFP intensities between Lipo2k/GFP siRNA and h(A2B3)-1/GFP siRNA, the optimal hyperbranched pBAE, h(A2B3)-1, exhibited much better silencing efficiency than Lipo2k using same concentration of siRNA (30 nM per well). This further proved that the h(A2B3)-1 can deliver siRNA to HeLa cells and effectively knockdown the target gene expression of the cells.

In comparison with commercial Lipo2k, h(A2B3)-1 has lower cytotoxicity and better siRNA knockdown efficiency, indicating a better safety profile and more potent siRNA transfection ability of h(A2B3)-1 polymer over commercial transfection reagent.

### Example 6: Gene Uptake and Silencing on Hard-to-Transfect Primary Neural Cells

### Isolation of Primary Neural Cells

Animal procedures were approved by the Institutional Animal Care and Use Committee guidelines of Nanyang Technological University (IACUC, NTU, protocol number: A190004)

Cortices were isolated from newborn (within 24 h) Sprague-Dawley rats followed by removal of the cerebrum and hippocampus, as well as the meninges. The cortices were digested using a mixture of 1.2 U papain, 40 μg/mL of DNase, and 0.24 mg/mL of L-cysteine that were dissolved in DMEM with 1% Pen/Strep, at 37° C., 5% CO₂ for 1 h.

The tissue digestion was stopped by adding DMEM Full medium (DMEM+10% FBS+1% Pen/Strep) digestion mix. All the remaining tissue pieces with medium were collected into a centrifuge tube. After centrifuging at 300 g for 5 min, the supernatant was discarded. Thereafter, the pellet was resuspended in DMEM Full medium and triturated through a 21 G and a 23 G needle for about 10 times sequentially. According to the established cell seeding density from our previous works (Zhang, Lin, & Chew, ACS Appl. Mater. Interfaces. 2021, 13, 55840-55850), cells from the cortices of 3 pups are seeded onto two PDL-(2.5 μg/mL) coated T75 flasks. After seeding, cells were cultured in DMEM Full medium at 37° C., 7.5% CO₂. Medium was refreshed every 3 days until day 10 when a monolayer of astrocytes was formed.

After 10 days of culture, microglia, which attach most loosely, were removed by shaking on an orbital shaker at 37° C., 200 rpm for 1 h. Thereafter, the supernatant was discarded, and the flasks were refilled with fresh DMEM Full medium for another round of shaking at 37° C., 200 rpm for 16 h to detach all OPCs. The remaining microglial were then eliminated by differential adhesion, which was performed by incubating the supernatant in an uncoated Petri dish for 30 min. After that, the supernatant was collected for centrifugation at 300 g for 5 min to obtain purified OPCs. Serum-free OPC culture medium which consisted of DMEM:Neurobasal (50:50), 2% B27, 1% Pen/Strep, Glutamax, ITS, 10 ng/mL biotin, 5 μg/mL NAC, and SATO (100 μg/mL BSA, 60 ng/mL progesterone, 16 μg/mL putrescine, and 400 ng/mL T3 and T4).

For gene silencing studies, OPCs were seeded at a density of 1×105 cells in each well of 24-well plates. All the wells were coated with 2.5 μg/mL of PDL and washed with PBS in advance. OPCs were maintained at 37° C., 7.5% CO2 incubator for 24 h before transfection.

Transfection was conducted by adding 15 w/w of h(A2B3)-1/siRNA into each well to obtain a final concentration of 100 nM of RNA. As the positive control, commercial transfection reagent, TKO (Mirus Bio LLC, US), was used, which comprised of 1:1 volume ratio of 50 μM siRNA following manufacturer's protocol. To evaluate gene silencing outcomes, RNA was extracted after 16 h of transfection.

The digestion of cortices for neuron culture was stopped by adding NB medium with 10% FBS into the digestion mix. After centrifuging at 300 g for 5 min, the cell pellet was resuspended in Neuron seeding medium (NB medium, 10% FBS, 2% B27 supplement, 1% N2 supplement, 1% Pen/Strep, 1% Glutamax) and triturated with a 21 G needle for 10 times to obtain uniform cell suspension. Thereafter, the suspension was passed through a 70 μm cell strainer to remove unbroken tissue clumps. The filtered cell suspension was then used for cell culture. Cells were seeded at a density of 2×10⁵/well on PDL-coated 24-well plates for gene silencing evaluation and 0.7×10⁵/13 mm PDL-coated coverslip for cell uptake evaluation. After seeding overnight, Neurobasal Full medium (NB medium, 2% B27 supplement, 1% Pen/Strep, 1% Glutamax) was used to replace the Neuron seeding medium. Neurons were maintained at 37° C., 5% CO₂ for 7 days before transfection, with medium change every 3 days. On Day 3, 10 μM of cytarabine was added to inhibit glial cells proliferation.

On Day 7, neurons were used for transfection. 10 w/w of h(A2B3)-1/siRNA or Cy5-labeled oligonucleotides (Cy5-ODN) was added into each well, to obtain a final concentration of 100 nM of siRNA. Commercial transfection reagent TKO (Mirus Bio LLC, US) was utilized as the positive control, which was used at 1:1 volume ratio to obtain a final concentration of 50 μM of siRNA. RNA was extracted after 16 h of transfection.

After 16 h of transfection, cells on coverslips were first washed with DPBS once and then fixed with 4% PFA for 15 min. Permeabilization and blocking were performed by incubating all samples in the same blocking buffer, which was composed of 1% BSA, 0.1% Triton in PBS for 1 h. All samples were incubated in the primary antibodies overnight at 4° C. After washing with PBS 3 times, all samples were incubated in secondary antibodies and DAPI at room temperature for 1 h before washing and mounting by covering the 13 mm round coverslip with stained cells on a 24 mm×50 mm coverslip. Details of the antibodies used are outlined in Table below. Thereafter, all samples were imaged under an inverted confocal microscope (Zeiss Airyscan Microscope-LSM800).

| Cell type | Primary antibodies | Secondary antibodies |
|---|---|---|
| Oligodendrocytes | Rabbit anti- | Goat anti- |
| | oligo2 (1:1000) | Rabbit 488 (1:1000) |
| | Rat anti- | Donkey anti- |
| | MBP (1:200) | Rat 555 (1:1000) |
| Cortical Neurons | Mouse anti- | Donkey anti- |
| | Tuj1 (1:1000) | Mouse 555 (1:1000) |

To investigate the gene expression level change after transfection, qPCR was conducted to compare the GAPDH expression level in different group settings. The groups were named as following: h(A2B3)-siGAPDH: h(A2B3)-1 polymers with siGAPDH. h(A2B3)-siNeg: h(A2B3)-1 with non-functional scrambled siRNA. TKO-siGAPDH and TKO-siNeg: commercial transfection reagent TKO with siGAPDH and siNeg respectively. RNA was extracted using RNeasy Mini Kit (Qiagen). Thereafter, 100 ng of RNA was reverse-transcribed into cDNA using LunaScript RT SuperMix Kit. A total of 10 μL of reaction mix for qPCR, which was composed of 0.25 μL of 10 μM primer pair, 1 μL cDNA, 4.75 μL deionized water and 5 μL Luna Universal qPCR Master Mix, was used for qPCR, using the QuantStudio 6 Flex QRT PCR Machine (Applied Biosystems). The following thermal cycle condition was used: 95° C. for 60 s for initial denaturation; 95° C. for 15 s followed by 60° C. for 30 s for 40 cycles) and followed by melt curve analyses. Three repeats for each sample and each gene were analyzed to obviate technical variation. All the primers sequences are listed in the table below. Since the primers have similar amplification efficiency, the comparative delta Ct method was used with β-actin as the housekeeping gene.

| Oligo | Forward | Reversal |
|---|---|---|
| name | chain (5′-3′) | chain (5′-3′) |
| GAPDH | ACCACAGTCCATGCCATCA | TCCACCACCCTGTTGCTGTA |
| primer | C [SEQ. ID NO: 1] | [SEQ. ID NO: 2] |
| b-Actin | ACGGTCAGGTCATCACTAT | TGCCACAGGATTCCATACCC |
| primer | CG [SEQ. ID NO: 3] | AG [SEQ. ID NO: 4] |

To explore the potential application of h(A2B3)-1 in neural system gene silencing, transfection study was conducted on primary neural cells, specifically cortical neurons, and oligodendrocyte progenitor cells (OPCs). These cells play key roles in nerve regeneration and remyelination after traumatic injuries and degeneration.

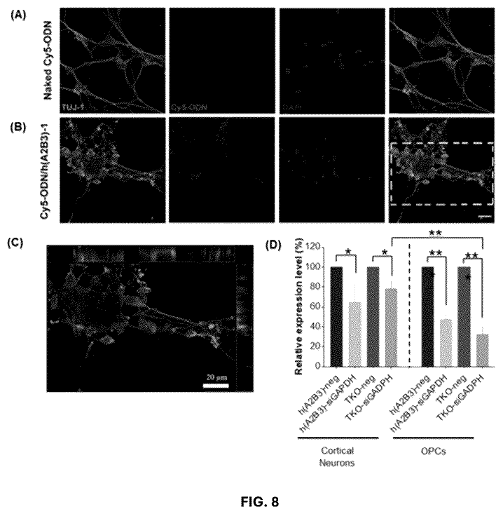

Cortical neurons were stained with TUJ1 and DAPI after transfection with h(A2B3)-Cy5-labeled oligonucleotide (Cy5-ODN) and the cellular uptake of the h(A2B3)-1 complexes were examined under confocal microscope. As shown in FIG. 8B, Cy5-ODN signals were detected within the cortical neurons as aggregates near the cell nuclei. Furthermore, the X—Z and Y—Z orthogonal views (FIG. 8C) indicate that the particles were indeed inside the cells and not merely attached onto the cell surfaces. On the other hand, when cells were transfected with bare Cy5-ODN, no Cy5 signals were detected (FIG. 8A). This indicated that h(A2B3)-1 complexes were well taken up by primary cortical neurons.

The gene silencing ability of h(A2B3)-1 complexes on primary cortical neurons and OPCs was evaluated by conducting cell transfection with commercial transfection reagent, TKO, as the positive control. We have previously shown that TKO effectively delivers small non-coding RNAs to primary neural cells (Diao, Low, Lu, & Chew, Biomaterials 2015, 70, 105-114; Milbreta et al., Mol. Ther. 2019, 27, 411-423; Zhang et al., Adv. Sci. 2019, 6, 1800808). As shown in FIG. 8D, h(A2B3)-1-mediated delivery of siGAPDH into primary rat cortical neurons resulted in significant silencing of GAPDH (^{˜}34.8% gene knockdown efficiency vs. siNEG treatment, p<0.05). On the other hand, the knockdown efficiency that was achieved by TKO-siGAPDH was only ^{˜}21.3%. In primary rat OPCs, similarly, significant gene knockdown of ^{˜}53.4% was observed in the presence of h(A2B3)-1. Comparatively, with the same concentration of siRNA, the knockdown efficiency was ^{˜}70% when TKO was used. This demonstrated that h(A2B3)-1 carrier could effectively knockdown target gene in primary cortical neurons and OPCs.

The results above showed that h(A2B3)-1 supported cell uptake of nucleic acids as well as downstream gene silencing. The extent of gene knockdown, however, was cell type dependent with OPCs showing better gene silencing outcomes regardless of transfection reagents used. One possibility for this difference may be the intrinsic difficulty to transfect neurons, due to the absence of cell cycle-dependent breakdown of the nuclear envelope in postmitotic cells (Huang & Chau, Mol. Pharmaceutics. 2021, 18, 377-385).

Interestingly, a trend of higher knockdown efficiency as observed in primary rat cortical neurons that were treated with h(A2B3)-1 vs. TKO. There are also many other gene carriers that have been reported in neural cells. For example, a type of amphiphilic 3-cyclodextrins, oligosaccharide-based molecules could achieve ^{˜}40% knockdown effect in a rat striatal cell line at 100 nM final concentration of siRNA, also at a mass ratio of 10 (carrier: siRNA). But noteworthy, postmitotic cells are always much more difficult to be transfected than cell lines (Godinho, Ogier, Darcy, O'Driscoll, & Cryan, Mol. Pharmaceutics. 2013, 10, 640-649). In very recent research, which used three charged lipid nanoparticles (LNP) with different zeta potential to transfect retinal ganglia neurons. Results came out with 40, 35, and 32% knockdown efficiency with increasing surface charge at a concentration of 45 pmol/5×10⁴ cells (Huang & Chau, Mol. Pharmaceutics. 2021, 18, 377-385). In comparison with our study, which used 12.5 pmol/5×10⁴ cell, the knockdown efficiency is quite similar (^{˜}34.8% in primary rat cortical neurons) even though three times lower amount of siRNA was delivered. As for OPCs, transfection with three particles made of polyornithine-based complexes, with different size and zeta potential at 100 nM siRNA, resulting in a range of decrease between 35% to 39% (Conejos-Sanchez et al., Nanoscale 2020, 12, 6285-6299). In comparison, the 53.4% knockdown efficiency of this h(A2B3)-1 polymer in our study reached an exciting degree which allows further in vivo applications.

### Example 7: Polymer Degradation and Post-Degradation Cytotoxicity

h(A2B3)-1 polymers were dissolved and incubated in sodium acetate buffer (pH 5.0) and PBS buffer (pH 7.4) at 37° C. to induce the polymer degradation. At incubation time points of 2, 4, 8, 20 and 24 h, degraded polymers were used to test the siRNA complexation ability using gel electrophoresis assay and evaluate their post-degradation cytotoxicity using MTT assay. Polymer at 0 h of incubation time was used as the control and treated as undegraded polymers.

pBAE is a class of cationic polymer which is biodegradable because of the hydrolysable ester bond along the polymer backbone. To study the polymer degradation of h(A2B3)-1, the polymers were incubated in PBS buffer (pH 7.4) and sodium acetate buffer (pH 5.0) for 2, 4, 8, 20, 24 h. After the designated time points of incubation, polymers were complexed with siRNA and tested for their siRNA condensation ability using gel electrophoresis assay. It is hypothesized that degraded polymers will lose their siRNA condensation ability because they are degraded into small molecules and cannot completely complex the nucleic acid.

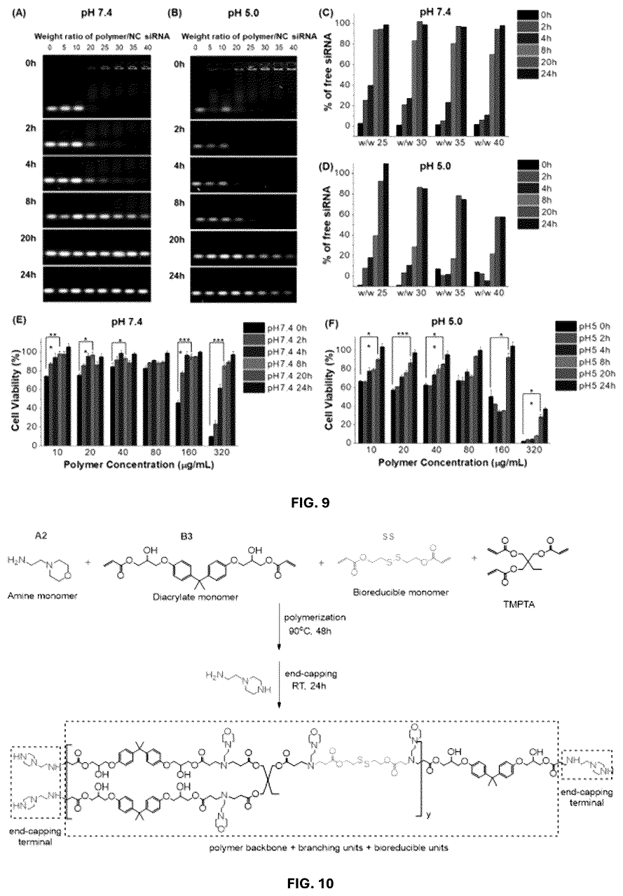

FIG. 9A-B show the gel retardation results of h(A2B3)-1/siRNA complexes after degradation. At pH 7.4, initially before incubation, h(A2B3)-1 could retard the siRNA migration at weight ratio of 20, however, it gradually lost its siRNA condensation ability with increasing incubation time. At 8, 20 and 24 h (and pH 7.4), siRNA bands were migrated down the gel at all weight ratios. At pH 5.0, h(A2B3)-1 also gradually lost its siRNA complexation efficiency with increasing incubation time. At 20 h and 24 h (and pH 5.0), the polymer could not retard the siRNA migration at all weight ratios. The amount of free siRNA at w/w 25-40 was quantified by measuring the band intensity of the migrated siRNA band and compared to control band of free siRNA at w/w 0. As shown in FIG. 9C-D, the amount of free siRNA (uncomplexed siRNA) increased over time at pH 7.4 and pH 5.0. At pH 7.4, more than 70% of free siRNA was observed at 8, 20 and 24 h, whereas lower percent of free siRNA was detected at pH 5.0. These results showed that the h(A2B3)-1 degraded faster at pH 7.4 than at pH 5 and the polymer degradation at pH 5.0 is more significant at longer incubation time e.g., 20 h and 24 h (60-90% of free siRNA) as compared to 8 h (<40% of free siRNA). These results are consistent with previous reports which investigated the hydrolysis kinetics of pBAE polymers at different pHs (Lynn & Langer, J. Am. Chem. Soc. 2000, 122, 10761-10768), and showed that this class of polymer hydrolyzed faster at pH 7.4 than at pH 5.1. Moreover, the polymer degradation might facilitate the siRNA release from the complexes, especially in the endosome, and subsequently enhance the siRNA transfection efficiency.

Furthermore, the post-degradation cytotoxicity of h(A2B3)-1 was studied in HeLa cells using MTT assay. As shown in FIG. 9E, at high concentration of polymers (160 and 320 μg/mL), the cytotoxicity of the degraded polymers decreased with increasing incubation time at pH 7.4. They exhibited significantly low cytotoxicity with over 80% of cell viability after 8 h of incubation (as compared to cell viability at 0 h), even if the polymer concentration was up to 320 μg/mL. At pH 5.0 (FIG. 9F), although h(A2B3)-1 degraded slower, it also showed reduced cytotoxicity over time and the low cytotoxicity of degraded polymers could be observed more significantly after longer incubation time e.g., 20 h. All these data confirmed that h(A2B3)-1 is biodegradable at both pH 7.4 and pH 5.0 with higher degradation rate at pH 7.4. Moreover, the degraded h(A2B3)-1 is highly cyto-compatible and non-toxic, demonstrating its great potential as safe gene delivery vectors.

The h(A2B3)-1 polymer showed good siRNA uptake and excellent siRNA transfection efficiency in the results above. However, the toxicity in the form of nanoparticles particularly at polymer concentrations above 40 μg/mL might become an issue to limit their application in the therapeutic field. Therefore, bioreducible disulfide bond was introduced into the polymer backbone to further improve the biocompatibility of h(A2B3)-1. It is well-known that the intracellular glutathione (GSH) concentration is about 2-10 mM whereas extracellular GSH level is only 1-10 μM. The disulfide bond can cleave spontaneously under the reductive environment with high GSH concentration while remain stable in extracellular environment with low GSH concentration.33, 47-49 Hence, introduction of disulfide bonds into the backbone of h(A2B3)-1 could make it become responsive to the intracellular reductive environment, induce spontaneous polymer degradation, decrease the cytotoxicity, and improve its biocompatibility.

The bioreducible h(A2B3)-1 was synthesized by the copolymerization of A2, B3, SS, and TMPTA via Michael addition approach (Scheme 1). Five disulfide-containing h(A2B3)-1, namely as S1, S2, S3, S4, and S5, with increasing mole percentage of SS monomer were prepared and characterized by 1H NMR (Figure S6). The measured mole percentage of disulfide content in S1 to S5 increased from 2.9% (S1), 6.8% (S2), 9.7% (S3), 19.8% (S4) to 26.6% (S5) respectively, which was calculated based on the integral ratios of the NMR spectra (Table S1). Next, their siRNA complexation ability was evaluated by gel electrophoresis assay. As shown in Figure S7, increasing SS % along the backbone of h(A2B3)-1 slightly affected the siRNA complexation. S1 with lowest disulfide percentage of 2.9% could retard the siRNA migration at weight ratio of 20 while S5 with highest disulfide percentage of 26.6% stopped the siRNA migration at weight ratio of 35.

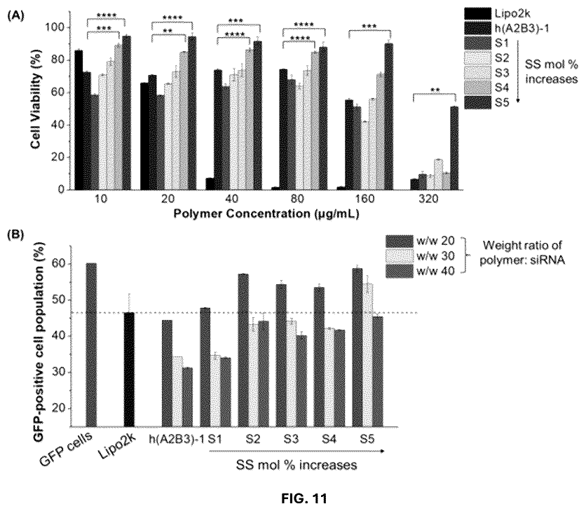

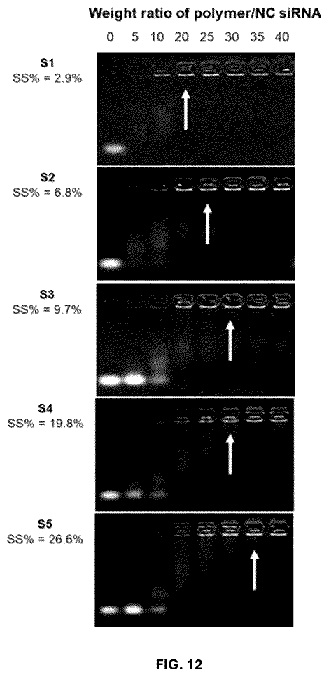

To investigate the effect of disulfide bond on biocompatibility, in vitro cytotoxicity of all five bioreducible h(A2B3)-1 was studied in HeLa cells, with Lipo2k and non-reducible h(A2B3)-1 as control. As shown in FIG. 11A, the polymer cytotoxicity decreased with increasing mol % of disulfide along the polymer backbone. In particular, S4 with disulfide percentage of 19.8% and S5 with disulfide percentage of 26.6% showed significant reduced cytotoxicity at polymer concentrations ranging from 10 to 320 μg/mL, as compared to their non-reducible h(A2B3)-1 without any disulfide linkage. S5 with disulfide percentage of 26.6% gave the best cyto-compatibility and maintained over 50% of cell viability even at the high polymer concentration up to 320 μg/mL.

The siRNA transfection efficiency of bioreducible h(A2B3)-1 was also investigated using GFP siRNA. Lipo2k and non-reducible h(A2B3)-1 were used as controls. As shown in FIG. 11B, the GFP silencing efficiency of the polymers was slightly decreased with increasing disulfide percentage from S1 (2.9%) to S5 (26.6%) as compared to non-reducible h(A2B3)-1. This might be because of the reduced siRNA complexation ability with higher disulfide content. Even though the silencing efficiency will be affected by the introduction of disulfide content, higher GFP silencing efficiency was observed in the bioreducible S1 to S4 at the weight ratios of 30 (and 40), as compared to Lipo2k. S5 with disulfide percentage of 26.6% could only achieve comparable GFP silencing efficiency with Lipo2k at weight ratio of 40.

Together with the cytotoxicity results, we concluded that the integration of disulfide features into the h(A2B3)-1 polymers can improve the biocompatibility but slightly reduce the siRNA transfection ability. With the aim to develop a siRNA delivery system which possesses good biocompatibility and efficient siRNA transfection efficiency simultaneously, S4 with disulfide percentage of 19.8% was chosen to be the optimal formulation among all bioreducible h(A2B3)-1 owing to its well balance performance between polymer cytotoxicity and siRNA silencing efficiency.

### Placeholder for Example

### Additional Aspects

1. A polymer of formula (I)

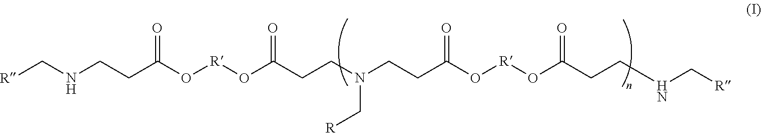

where:
each R is selected from (CH₂)₃OH and

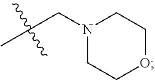 [SMILES: *CN1CCOCC1]

each R′ is selected from

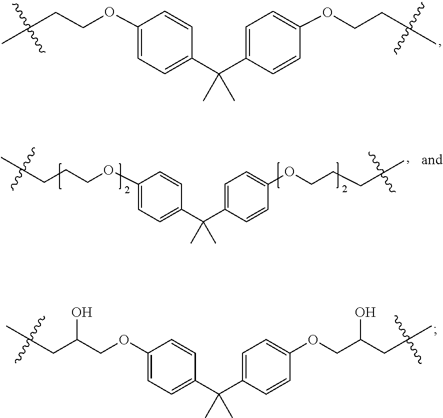

each R″ is selected from

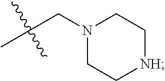 [SMILES: *CN1CCNCC1]

where each wiggly line represents the point of attachment to the polymer.
2. A polymer of formula (II)

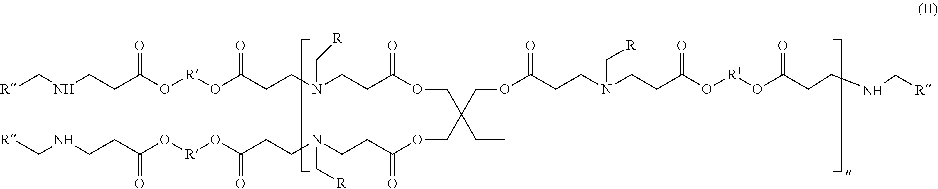

where:
each R is selected from (CH₂)₃OH and

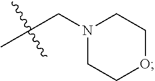 [SMILES: *CN1CCOCC1]

each R′ is selected from

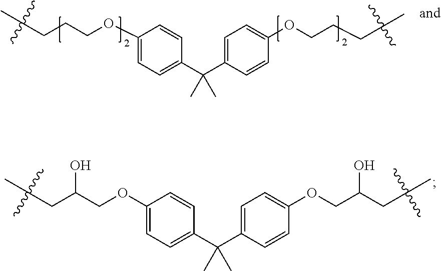

each R″ is selected from

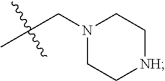 [SMILES: *CN1CCNCC1]

where each wiggly line represents the point of attachment to the polymer.
3. The polymer according to Clauses 1 and 2, wherein the polymer of formula (I) and (II), is selected from the list:

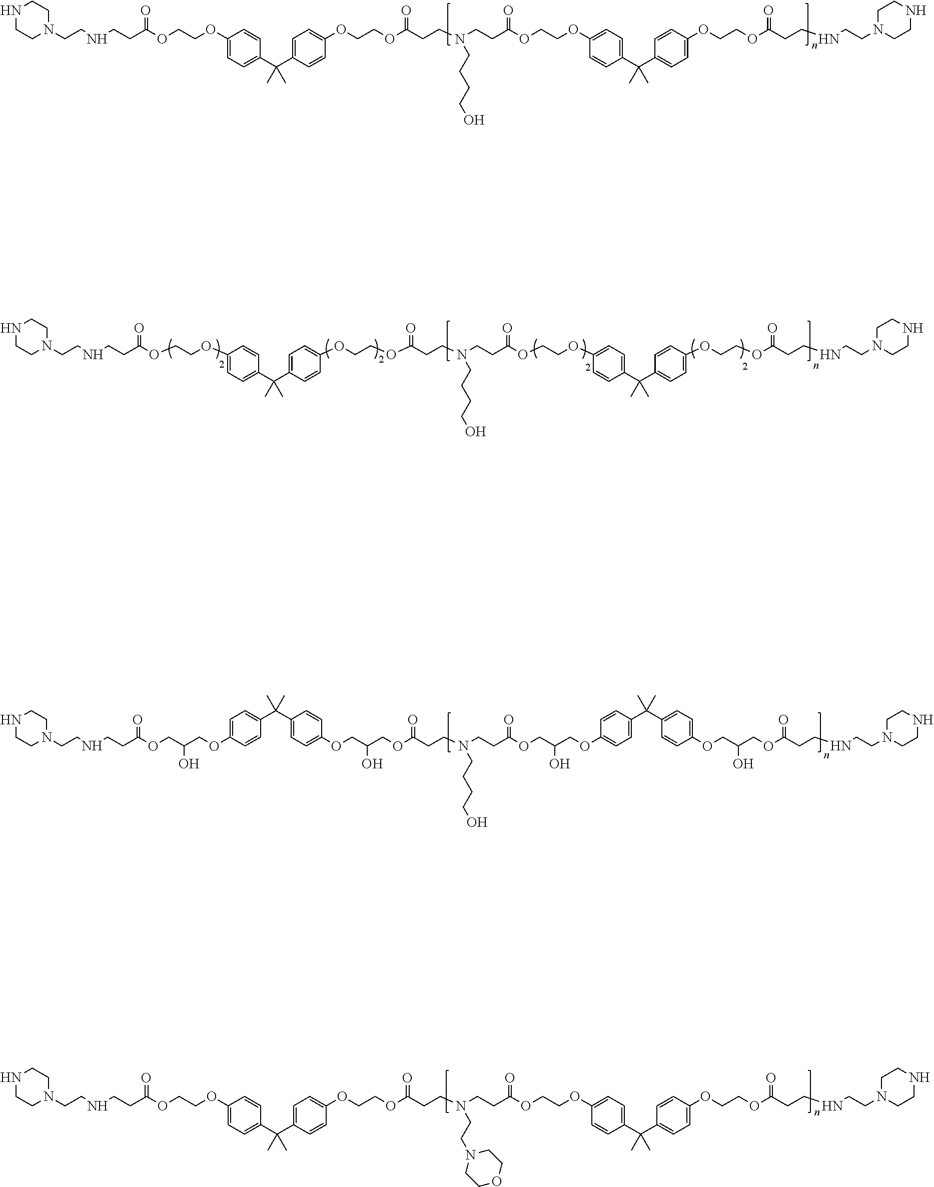

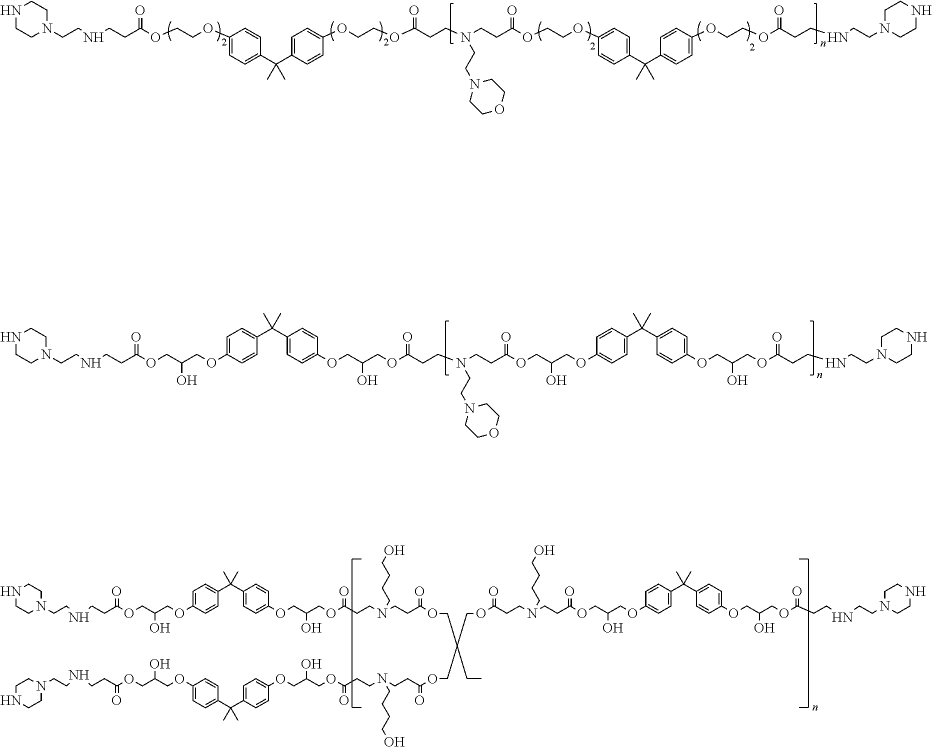

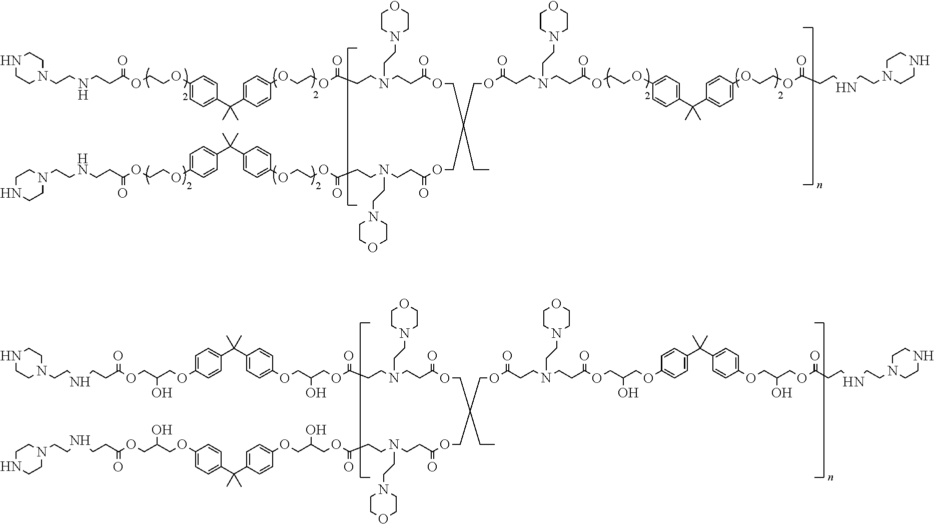

4. A method of preparing a polymer of formula (I) by reacting together a diacrylate monomer of formula (B) and an amine monomer of formula (A) via Michael addition reaction to obtain product (i); and combining product (i) with an end-capping monomer of formula (C) via Michael addition reaction.

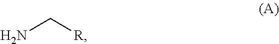

where R is selected from (CH₂)₃OH and

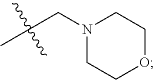

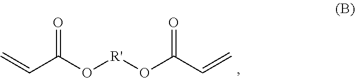

where R′ is selected from

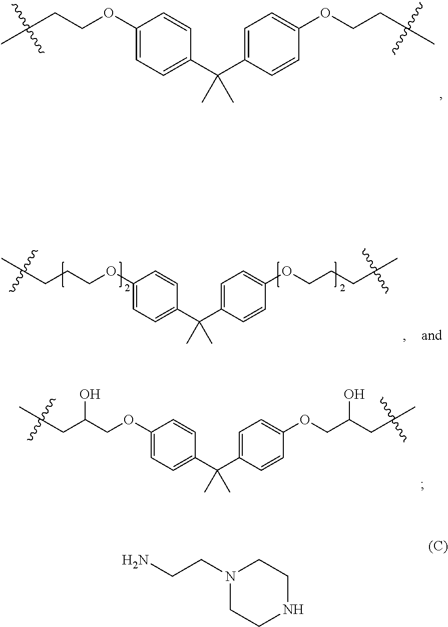

5. A method of preparing a polymer of formula (II) by reacting together a diacrylate monomer of formula (B), an amine monomer of formula (A) and a triacrylate monomer of formula (D) via Michael addition reaction to obtain product (ii); and combining product (ii) with an end-capping monomer of formula (C) via Michael addition reaction.

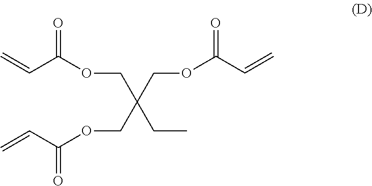 [SMILES: C=CC(=O)OCC(CC)(COC(=O)C=C)COC(=O)C=C]

6. A composition comprising a polymer of formula (I) or (II) according to any one of Clauses 1 to 2, and a nucleic acid biological agent. In certain embodiment, the nucleic acid biological agent is long noncoding (Inc) ribonucleic acid (RNA) (lncRNA), small non-coding (snc) RNA (sncRNA), Piwi-interacting RNA (piRNA), interfering RNA (RNAi), small interfering RNA (siRNA), repeat associated small interfering RNA (rasiRNA), microRNA (miRNA), and/or antisense oligonucleotide consisting of 21- to 23-nucleotide double stranded RNA.
7. A method of preparing composition comprising a polymer of formula (I) or (II) according to any one of Clauses 1 to 2, and a nucleic acid biological agent via electrostatic complexation approach.
8. A method of delivering long noncoding (Inc) ribonucleic acid (RNA) (lncRNA), small non-coding (snc) RNA (sncRNA), Piwi-interacting RNA (piRNA), interfering RNA (RNAi), small interfering RNA (siRNA), repeat associated small interfering RNA (rasiRNA), microRNA (miRNA), and/or antisense oligonucleotide to a cell by contacting the cell with a composition comprising a polymer of formula (II) according to any one of Clause 2, and a siRNA.
9. A method of silencing target gene expression in a cell, the method comprising administering a composition comprising a polymer of formula (II) according to any one of Clauses 2, and an effective amount of a long noncoding (Inc) ribonucleic acid (RNA) (lncRNA), small non-coding (snc) RNA (sncRNA), Piwi-interacting RNA (piRNA), interfering RNA (RNAi), small interfering RNA (siRNA), repeat associated small interfering RNA (rasiRNA), microRNA (miRNA), and/or antisense oligonucleotide.
10. Uses of a composition comprising a polymer of formula (II) according to one of Clause 2, and a long noncoding (Inc) ribonucleic acid (RNA) (lncRNA), small non-coding (snc) RNA (sncRNA), Piwi-interacting RNA (piRNA), interfering RNA (RNAi), small interfering RNA (siRNA), repeat associated small interfering RNA (rasiRNA), microRNA (miRNA), and/or antisense oligonucleotide for gene delivery and gene silencing in easy-to-transfect cells or hard-to-transfect cells.
11. A pharmaceutical composition comprising a polymer of formula (II) according to any one of Clause 2, and a pharmaceutically acceptable long noncoding (Inc) ribonucleic acid (RNA) (lncRNA), small non-coding (snc) RNA (sncRNA), Piwi-interacting RNA (piRNA), interfering RNA (RNAi), small interfering RNA (siRNA), repeat associated small interfering RNA (rasiRNA), microRNA (miRNA), and/or antisense oligonucleotide for use in medicine.
12. A kit of parts comprising:
(a) Amine monomers (A), diacrylate monomers (B), or a combination of these, as well as poly(β-amino esters); and
(b) Vials, solvents, buffers, multi-well plates, salts, and agents described herein (e.g., polynucleotides or siRNA); and
(c) Detailed instructions on how to prepare the inventive end-modified polymers.
(d) Detailed instructions on how to prepare the composition comprising the inventive end-modified polymers and the nucleic acid biological reagent.
13. The kit of parts according to Clause 12, wherein the kit is further customized to produce end-modified polymers with specific desired properties for a specific use.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches

1. A polymer the formula: 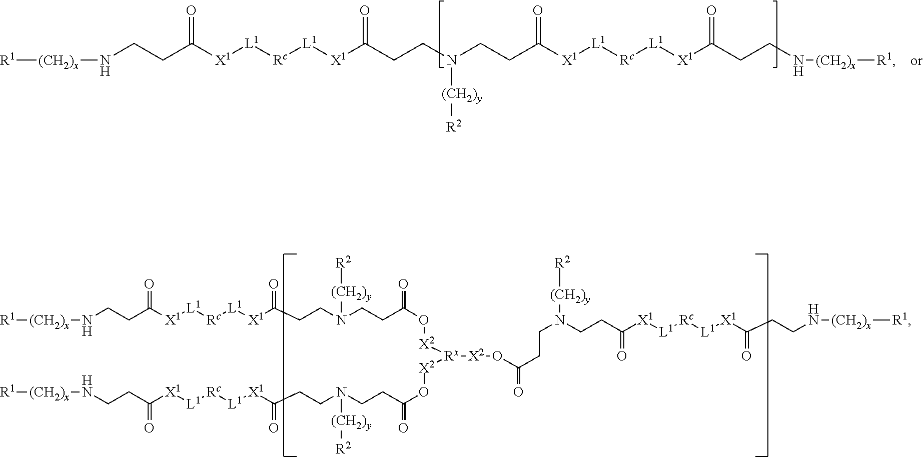
wherein:
R¹ is NH₂, NH(C₁₋₆alkyl), N(C₁₋₆alkyl)₂, C₁₋₈heterocyclyl, or C₁₋₈heteroaryl;
X is 1, 2, 3, 4, or 5;
X¹ is O or NH;
L¹ is null or a C₂₋₂₀aliphatic group;
R^{c} is in each case independently a C₂₋₂₀aliphatic group;
R² is OH, NH₂, NH(C₁₋₆alkyl), N(C₁₋₆alkyl)₂, C₁₋₈heterocyclyl, or C₁₋₈heteroaryl;
Y is 1, 2, 3, 4, or 5;
X² is null, CH₂ or CH₂CH₂; and
R^{x} is N or a C₁₋₆aliphatic group.

2. The polymer of claim 1, wherein R¹ and R² are not the same, and X¹ is O.

3. The polymer of claim 1, wherein the polymer has the formula: 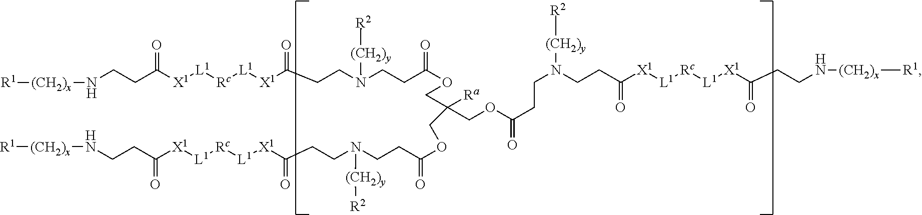
wherein R^{a} is H, CH₃, or CH₂CH₃.

4. The polymer of claim 1, obtained by:
(a) synthesizing an uncapped polymer by a providing a mixture comprising a compound of Formula (1):
R²—(CH₂)_{y}—NH₂ [Formula (1)];
a compound of Formula (2) 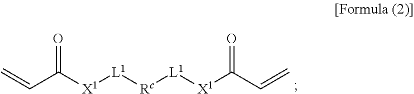
and
(b) reacting the uncapped polymer with a compound of Formula (4):
R¹—(CH₂)ₓ—NH₂ [Formula (4)].

5. The polymer of claim 4, wherein the mixture further comprises a compound of Formula (3):

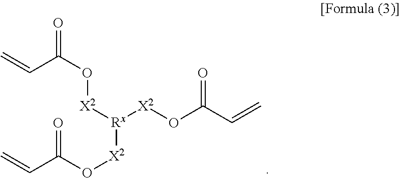

6. The polymer of claim 4, wherein the mixture further comprises a compound of Formula (4): 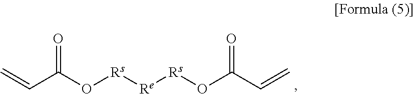
wherein R^{s} is a C₂₋₆aliphatic group and R^{e} is a bioerodible group.

7. The polymer of claim 6, wherein R^{s} is in each case CH₂CH₂, C(CH₃)₂CH₂, CH(CH₃)CH₂, CH₂CH₂CH₂, C(CH₃)₂CH₂CH₂, or CH(CH₃)CH₂CH₂, and R^{e} is S—S, S—CH₂—S, S—C(CH₃)₂—S—, or O—C(═O)—C(═O)—O.

8-10. (canceled)

11. The polymer of claim 4, wherein the compound of Formula (1) and Formula (2) are present in the mixture in a molar ratio from 1.00-2.00:1.

12. The polymer of claim 5, wherein the compound of Formula (2) and Formula (3) are present in the mixture in a molar ratio from 1:0.1-0.75.

13. The polymer of claim 6, wherein the compound of Formula (2) and Formula (5) are present in the mixture in a molar ratio from 1:0.01-0.50.

14. (canceled)

15. The polymer of claim 1, wherein R¹ is a C₄₋₈heterocycle.

16-20. (canceled)

21. The polymer of claim 1 any of claims 1-19, wherein L¹ has the formula: 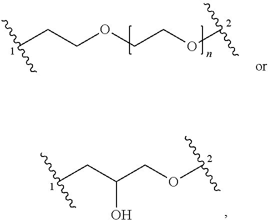
wherein n is 0-8, and wavy line 1 represents the point of connection to X¹ and wavy line 2 represents the point of connection of R^{c}.

22. The polymer of claim 1, wherein R^{c} is independently chosen from: 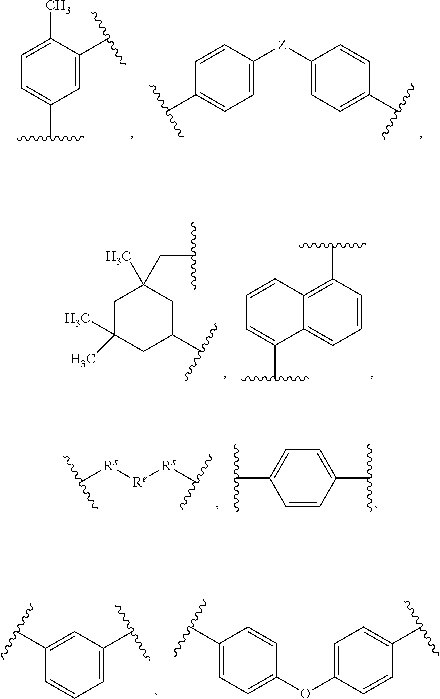
wherein Z is null, CH₂, C(CH₃)₂, O, or NH, and R^{e} is a bioerodible group, and R^{s} is a C₂₋₆aliphatic group.

23. (canceled)

24. The polymer of any of claims 1-23, wherein R^{c} is independently:

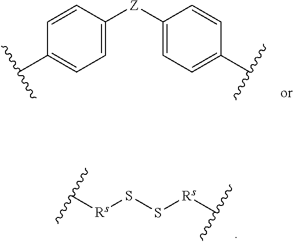

25. The polymer of claim 22, wherein R^{c} is independently: 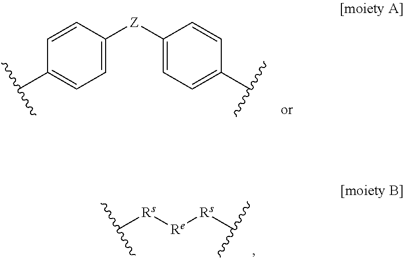
wherein the ratio of [moiety A]:[moiety B] is from 1:0.01-0.5.

26. (canceled)

27. The polymer of claim 1, wherein R² is H, OH, NH₂, NHCH₃, N(CH₃)₂, C₄₋₈heterocyclyl, or C₃₋₆heteroaryl.

28-30. (canceled)

31. The polymer of claim 1, wherein R^{x} is N or a group having the formula: 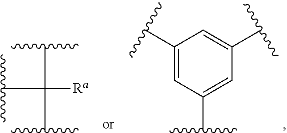
wherein R^{a} is H, CH₃, or CH₂CH₃.

32. (canceled)

33. A composition comprising the polymer of claim 1 and a nucleic acid.

34-38. (canceled)

39. A method of providing a nucleic acid to a subject in need thereof, comprising administering to the subject the composition according to claim 33.

40-50. (canceled)

51. A method of making a composition, comprising mixing the polymer of claim 1 any of claims 1-32 and a nucleic acid.

52. (canceled)
